SEQUENCE LISTING (20 sequences)

SEQ ID NO: 1 - DNA, 630 bp, Hepatitis C Virus
  CDS (43)...(630)
ggtgcttgcgagtgccccgggaggtctcgtagaccgtgcaccatgagcacgaatcctaaacctcaaagaaaaaccaaacgtaacaccaaccgtcgcccacaggacgttaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcacgtcggcccaagggcaggaactgggctcagcccgggtatccttggcccctctatggcaatgagggttgcgggtgggcgggatggctcctgtcccccagtggctctcggcctagttggggccccaacgacccccgacgtaggtcgcgcaatttgggtaaggtcatcgatacccttacgtgcggcttcgtcgatctcatggggtacataccgctcgtcggcgcccctcttagaggcgctgccagggccctggcgcatggcgtccgggttctggaagacggtgtgaactatgcaacaggtaaccttcctggttgctctttctctatcttccttctggccctgctctcttgcctgactgtgcccgcttcagcctaccaagtgcgcaac

SEQ ID NO: 2 - DNA, 60 bp, Hepatitis C Virus
  CDS (1)...(60): Corresponds to aa 125 to 144 of SEQ ID. No. 1
acccttacgtgcggcttcgtcgatctcatggggtacataccgctcgtcggcgcccctctt

SEQ ID NO: 3 - DNA, 18 bp, Hepatitis C Virus
  CDS (1)...(18): Corresponds to aa 161-166 of SEQ ID. No. 1
ggtgtgaactatgcaaca

SEQ ID NO: 4 - DNA, 1900 bp, Human
  misc_feature (1)...(1900): n = A,T,C or G
cgtcttcgggacgcgcccgctcttcgcctttcgctgcagtccgtcgatttctttctccaggaagaaaaatggcatccgttgcagttgatccacaaccgagtgtggtgactcgggtggtcaacctgcccttggtgagctccacgtatgacctcatgtcctcagcctatctcagtacaaaggaccagtatccctacctgaagtctgtgtgtgagatgscagagaacggtgtgaagaccatcacctccgtggccatgaccagtgctctgcccatcatccagaagctagagccgcaaattgcagttgccgatacctatgcctgtaaggggctagacaggattgaggagagactgcctattctgaatcagccatcaactcagattgttgccaatgccaaaggcgctgtgactggggcaaaagatgctgtgacgactactgtgactggggccaaggattctgtngccagcacgatcacaggggtgatggacaagaccaaaggggcagtgactggcagtgtggagaagaccaagtctgtggtcagtggcagcattaacacagtcttggggagtcggatgatgcagctcgtgagcagtggcgtagaaaatgcactcaccaaatcagagctgttggtagaacagtacctccctctcactgaggaagaactagaaaaagaagcaaaaaaagttgaaggatttgatctggttcagaagccaagttattatgttagactgggatccctgtctaccaagcttcactcccgtgcctaccagcaggctctcagcagggttaaagaagctaagcaaaaaagccaacagaccatttctcagctccattctactgttcacctgattgaatttgccaggaagaatgtgtatagtgccaatcagaaaattcaggatgctcaggataagctctacctctcatgggtagagtggaaaaggagcattggatatgatgatactgatgagtcccactgtgctgagcacattgagtcacgtactcttgcaattgcccgcaacctgactcagcagctccagaccacgtgccacaccctcctgtccaacatccaaggtgtaccacagaacatccaagatcaagccaagcacatgggggtgatggcaggcgacatctactcagtgttccgcaatgctgcctcctttaaagaagtgtctgacagcctcctcacttctagcaaggggcagctgcagaaaatgaaggaatctttagatgacgtgatggattatcttgttaacaacacgcccctcaactggctggtaggtcccttttatcctcagctgactgagtctcagaatgctcaggaccaaggtgcagagatggacaagagcagccaggagacccagcgatctgagcataaaactcattaaacctgcccctatcactagtgcatgctgtggccagacagatgacaccttttgttatgttgaaattaacttgctaggcaaccctaaattgggaagcaagtagctagtataaaggccctcaattgtagttgtttccagctgaattaagagctttaaagtttctggcattagcagatgatttctgttcacctggtaagaaaagaatgataggcttgtcagagcctatagccagaactcagaaaaaattcaaatgcacttatgttctcattctatggccattgtgttgcctctgttactgtttgtattgaataaaaacatcttcatgtgggctggggtagaaactggtgtctgctctggtgtgatctgaaaaggcgtcttcactgctttatctcatgatgcttgcttgtaaaacttgattttagtttttcatttctcaaataggaatactacctttgaattcaataaaattcactgcaggatagaccagttnagnagcaaacanncangtacacnnaaganac

SEQ ID NO: 5 - protein, 437 aa, Human
  VARIANT (1)...(437): Xaa = Any Amino Acid
MASVAVDPQPSVVTRVVNLPLVSSTYDLMSSAYLSTKDQYPYLKSVCEMXENGVKTITSVAMTSALPIIQKLEPQIAVADTYACKGLDRIEERLPILNQPSTQIVANAKGAVTGAKDAVTTTVTGAKDSVASTITGVMDKTKGAVTGSVEKTKSVVSGSINTVLGSRMMQLVSSGVENALTKSELLVEQYLPLTEEELEKEAKKVEGFDLVQKPSYYVRLGSLSTKLHSRAYQQALSRVKEAKQKSQQTISQLHSTVHLIEFARKNVYSANQKIQDAQDKLYLSWVEWKRSIGYDDTDESHCAEHIESRTLAIARNLTQQLQTTCHTLLSNIQGVPQNIQDQAKHMGVMAGDIYSVFRNAASFKEVSDSLLTSSKGQLQKMKESLDDVMDYLVNNTPLNWLVGPFYPQLTESQNAQDQGAEMDKSSQETQRSEHKTH

SEQ ID NO: 6 - protein, 31 aa, Artificial Sequence
  branched peptide containing residues 5-27 of HCV core protein
XKPQRKTKRNTXRRPQDVKFPGGKKKKKKKA

SEQ ID NO: 7 - DNA, 11 bp, Artificial Sequence
  oligonucleotides used to construct HCV core protein deletion plasmids
gctgagatcta

SEQ ID NO: 8 - DNA, 29 bp, Artificial Sequence
  oligonucleotides used to construct HCV core protein deletion plasmids
gtaaccttcctggttgctcttgagatcta

SEQ ID NO: 9 - DNA, 17 bp, Artificial Sequence
  oligonucleotides used to construct HCV core protein deletion plasmids
gtaacctttgagatcta

SEQ ID NO: 10 - DNA, 18 bp, Artificial Sequence
  oligonucleotides used to construct HCV core protein deletion plasmids
ctggcgcattgagatcta

SEQ ID NO: 11 - DNA, 28 bp, Artificial Sequence
  oligonucleotides used to construct HCV core protein deletion plasmids
ctggcccatggtgttaactatgcaacag

SEQ ID NO: 12 - DNA, 31 bp, Artificial Sequence
  oligonucleotides used to construct HCV core protein deletion plasmids
ctggcccatggcgtccgggttctggaagacg

SEQ ID NO: 13 - DNA, 37 bp, Artificial Sequence
  oligonucleotides used to construct HCV core protein deletion plasmids
cgatagaggcgctgccagggccctggcgtgagatcta

SEQ ID NO: 14 - DNA, 52 bp, Artificial Sequence
  HCV1 oligonucleotide for plasmid construction
catggggtacatagcgctcgtcggcgccgccttaagaggcgctgcgagggcc

SEQ ID NO: 15 - DNA, 36 bp, Artificial Sequence
  HCV2 oligonucleotide for plasmid construction
ctagagagcgcaagacgccccgcgtcaccggcggcg

SEQ ID NO: 16 - DNA, 26 bp, Artificial Sequence
  primer derived from GBV-B, nucleotides 428-448, for plasmid construction
ggagatctcgtagaccgtagcacatg

SEQ ID NO: 17 - DNA, 38 bp, Artificial Sequence
  primer derived from GBV-B, nucleotides 842-868, for plasmid construction
ggggatccctagtggacaccgaaccaaccagtagccca

SEQ ID NO: 18 - DNA, 36 bp, Artificial Sequence
  primer derived from GBV-B, nucleotides 1003-1029, for plasmid construction
ggggatcctcagatcacacaaccaggctcgtgtagg

SEQ ID NO: 19 - DNA, 27 bp, Artificial Sequence
  primer derived from GBV-B, nucleotides 1618-1639, for plasmid construction
gggtactctagagtgataggcctggtc

SEQ ID NO: 20 - DNA, 49 bp, Artificial Sequence
  primer derived from GBV-B for plasmid construction
ctagagagcgcaagacgccgcgggtcaccggtggctctcgcaatcttgg

(19) United States
(12) Patent Application Publication
(10) Pub. No.: US 2002/0115066 A1
(43) Pub. Date: Aug. 22, 2002

(54) Viral therapeutics

(76) Inventors: Ralph Graham Hope, Jordanhill (GB); John McLauchlan, Bishopbriggs (GB)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/973,322

(22) Filed: Oct. 9, 2001

Related U.S. Application Data
(63) Continuation-in-part of application No. 09/201,916, filed on Dec. 1, 1998.

(30) Foreign Application Priority Data
Nov. 26, 1998 (GB) 9825951.8

(51) Int. Cl. C12Q 1/70; A61K 39/12; C07K 14/02; C07H 21/04

(52) U.S. Cl. 435/5; 424/186.1; 530/350; 536/23.72

(57) ABSTRACT

A method for identifying a substance for affecting a viral infection is described. The method comprises providing a lipid globule targeting sequence, as a first component; providing a lipid globule, as a second component; contacting the two components with a substance to be tested under conditions that would permit the two components to interact in the absence of the substance; and determining whether the substance disrupts the interaction between the first and second components; where the targeting sequence comprises a hepatitis C virus (HCV) core protein or a fragment, derivative, variant or homologue thereof.

### RELATED APPLICATIONS

This application is a Continuation-in-Part of U.S. application Ser. No. 09/201,916 filed Dec. 1, 1998, which claims priority to Great Britain patent application No. 9825951.8, filed Nov. 26, 1998, the disclosures of which are herein incorporated by reference in their entireties.

### BACKGROUND OF THE INVENTION

1. Field of the Invention

This invention relates to substances capable of modulating the interaction between viral proteins capable of binding to intracellular lipid globules, cellular adipocyte-specific differentiation-related protein and intracellular lipid globules. The invention also relates to assays for identifying such substances and the use of these substances in affecting viral infection.

2. Description of the Related Art

Hepatitis C virus (HCV) is a major causative agent of chronic hepatitis and liver disease. It is estimated that, worldwide, approximately 300 million individuals are infected with the virus, 20% of whom are likely to develop mild to severe liver disease or carcinoma. Apart from the risk of succumbing to the long term effects of infection, these individuals also represent a large reservoir of virus for future transmissions. To date, the only widely used therapy for HCV is treatment with interferon. However, sustained response is achieved in only about 20% of cases. Moreover, no vaccine currently exists to protect against infection. Since growth of the virus has not been possible to date in tissue culture systems, very little is known also about the molecular events which occur during viral replication.

The core protein of HCV is predicted to constitute the capsid of virus particles. From various studies, expression of this protein results in a range of effects on intracellular processes, including a decrease in transcription of genes from HBV and HIV and alterations to apoptosis. There is also evidence from a study on transgenic mice that liver-specific expression of core may be linked to the development of steatosis (fatty liver), a condition commonly found in HCV-infected individuals which is characterized by the accumulation of fat deposits within hepatocytes. Thus, core protein may also influence lipid metabolism within the liver. Other results from studies on human sera suggest that HCV virus particles are found associated with lipoprotein particles which are produced by the liver. It has also been shown that HCV core protein associates with lipid droplets within cells (Barba et al., 1997; Moradpour et al., 1996). The droplets are storage compartments for both triacylglycerols and cholesterol esters which can be used as substrates for oxidation in mitochondria and for the formation of membranes. In specialized cells, stored cholesterol is used for steroid hormone synthesis.

Within the liver, lipid droplets also function as a site for storage of precursors of the lipid which is secreted from this organ in the form of lipoprotein particles. Although lipid droplets were identified several decades ago and they can be readily detected by staining methods, very little is known about the processes of assembly, storage and disassembly within the cell.

### SUMMARY OF THE INVENTION

We have surprisingly shown that expression of HCV core protein and its resultant association with intracellular lipid droplets results in the loss of a protein, termed adipocyte-specific differentiation-related protein (ADRP), from the droplets. ADRP has been found to associate with lipid droplets in a range of cell types and in certain organs. It has been proposed that ADRP may be required for maintenance of lipid droplets within cells, however the precise function of the protein has not been identified. Furthermore, progressive increases in core expression result in diminishing amounts of ADRP to undetectable levels. We have also identified regions of the HCV core protein which are required for both lipid globule association and down-regulation of ADRP levels. Without wishing to be bound by theory, we believe that displacement of ADRP by HCV core protein may be a factor involved in HCV infection. Thus, the interaction of HCV core with intracellular lipid globules and associated displacement of ADRP represents a target for therapies designed to prevent or reduce the effects and/or progression of HCV infection.

One embodiment of the present invention relates to a method for identifying a substance capable of affecting a viral infection. The method comprises providing a lipid globule targeting sequence, as a first component; providing a lipid globule, as a second component; contacting the two components with a substance to be tested under conditions that would permit the two components to interact in the absence of the substance; and determining whether the substance disrupts the interaction between the first and second components.

Preferably, the targeting sequence comprises a hepatitis C virus (HCV) core protein or a fragment thereof, or a GB virus-B core protein or a fragment thereof. More preferably, the targeting sequence further comprises variants or homologues of the hepatitis C virus (HCV) core protein or the GB virus-B core protein.

In a variation, to the method, the substance to be tested is administered to a cell, the lipid globule targeting sequence is expressed in the cell, and the lipid globule is a natural constituent of the cell. The lipid globule targeting sequence may be naturally or recombinantly expressed in the cell.

In a further variation to the method, a virus is administered to a cell in the absence of the substance which has been determined to disrupt the interaction between the first and second components. The virus is also administered to the cell in the presence of the substance. It is then determined if the substance reduces or abolishes the susceptibility of the cell to viral infection or the effects of viral infection. The cell is preferably a liver cell.

In another preferred mode of the invention, the lipid globule targeting sequence comprises amino acids of the HCV core protein selected from the group consisting of 125 to 144, 161 to 166 and the combination thereof.

The viral infection is preferably a hepatitis infection or other viral infection of the human or animal liver.

Another aspect of the invention relates to the substance identified by the above methods. Preferably, the substance has not previously been known to affect viral infection.

Another method for identifying a substance for treating or preventing a viral infection, comprises: administering the substance to a mammalian cell; and identifying whether the administration of the substance upregulates expression of adipocyte-specific differentiation related protein (ADRP) in the mammalian cell.

Another aspect of the invention relates to a substance capable of disrupting an interaction between a lipid globule targeting sequence and a lipid globule for use in affecting a viral infection, wherein the targeting sequence comprises a hepatitis C virus (HCV) core protein or a fragment thereof, or a GB virus-B core protein or fragment thereof.

A further aspect of the invention relates to a polypeptide comprising a lipid globule targeting sequence for use in preventing or treating a viral infection, wherein the targeting sequence comprises an HCV core protein or a fragment, variant or homologue thereof, or a GB virus-B core protein or a fragment, variant or homologue thereof.

Preferably, the polypeptide comprises the targeting sequence comprising amino acids of the HCV core protein selected from the group consisting of 125 to 144, 161 to 166 and the combination thereof.

Another variation to the present invention relates to a pharmaceutical composition comprising the polypeptide and a pharmaceutically acceptable carrier or diluent. Further, a method is provided for treating or preventing a viral infection comprising administering an effective amount of the pharmaceutical composition.

Another method for treating or preventing a viral infection is provided, comprising administering an effective amount of a pharmaceutical composition comprising the substance identified as disrupting the interaction between the first and second components, and a pharmaceutically acceptable carrier or diluent.

A further method is provided for treating or preventing a viral infection comprising administering an effective amount of a pharmaceutical composition comprising the substance identified as upregulating expression of adipocyte-specific differentiation related protein (ADRP) in a mammalian cell.

In accordance with another embodiment of the present invention, a polynucleotide is provided encoding a polypeptide comprising a lipid globule targeting sequence for use in treating or preventing a viral infection.

Another preferred embodiment of the present invention relates to a method for determining whether a test substance is capable of treating or preventing a viral infection. The method comprises: providing a lipid globule targeting sequence, as a first component, the targeting sequence comprising a hepatitis C virus (HCV) core protein or a fragment thereof, or a GB virus-B core protein or fragment thereof; providing a lipid globule, as a second component; incubating the first and second components with the test substance under conditions that would permit the first and second components to interact with one another in the absence of the test substance; and determining whether the test substance disrupts the interaction between the first and second components.

### BRIEF DESCRIPTION OF THE DRAWINGS

FIG. 1 shows Western blots probed with antibodies to HCV core protein.

FIG. 2 shows confocal microscopy images of the intracellular localization of core proteins and lipid droplets.

FIG. 3 shows confocal microscopy images of cells illustrating the effect of expression of HCV core proteins on the ability to detect ADRP in BHKC13 cells.

FIG. 4 shows confocal microscopy images of cells illustrating the effect of expression of HCV core protein on the abundance of ADRP.

FIG. 5 shows Western blots probed with antibodies to HCV core protein and adipophilin.

FIG. 6 shows Western blots probed with antibodies to HCV core protein.

FIG. 7 shows the amino acid sequence comparison between the predicted core proteins of HCV and GBV-B.

### DETAILED DESCRIPTION OF THE PREFERRED EMBODIMENT

Although in general the techniques mentioned herein are well known in the art, reference may be made in particular to Sambrook et al., Molecular Cloning, A Laboratory Manual (1989) and Ausubel et al., Current Protocols in Molecular Biology (1995), John Wiley & Sons, Inc.

A. Proteins/Polypeptides

The term “protein” includes single-chain polypeptide molecules as well as multiple-polypeptide complexes where individual constituent polypeptides are linked by covalent or non-covalent means. The term “polypeptide” includes peptides of two or more amino acids in length, typically having more than 5, 10 or 20 amino acids.

1. Lipid Globule Targeting Sequences

The term “lipid globule targeting sequence” means an amino acid sequence which is capable of association with a lipid globule, preferably a biologically occurring lipid globule such as an intracellular lipid globule as found in adipocytes or a secreted lipid globule as found in mammalian milk. In addition, the lipid globule targeting sequence is preferably capable of association with a lipid globule when linked to a protein of interest such that the protein of interest is also associated with the lipid globule by virtue of being linked to the targeting sequence. Lipid globule association may take place within a non-cellular and/or extra-cellular environment, such as in an apparatus—for example a tube or vat. Alternatively, it may take place in a cellular environment where the expressed targeting sequence is directed to intracellular lipid droplets or the membranes of such droplets.

The ability of an amino acid sequence to associate with/target lipid globules can be assessed either in vitro or in vivo. For example, a candidate targeting sequence may be added to a dispersion of lipid globules (such as a mixture of phospholipid and triacylglycerol) in an aqueous solvent, the mixture sonicated and the degree of partition between aqueous and lipid phases determined by fractionation. Typically fractionation of the mixture would involve increasing the density of the solution with sorbitol or sodium bromide and ultracentrifuging the solution. The lipid complexes migrate to the top of the centrifuge tube and this upper lipid layer is then examined for candidate targeting sequence. Preferably, a suitable lipid globule targeting sequence should partition at least 50:50 lipid:aqueous phase, more preferably at least 75:25, 80:20 or 90:10.

Another suitable test may involve introducing a polynucleotide encoding a candidate sequence, optionally linked to a protein of interest, into a milk-producing cell in culture and determining whether, the targeting sequence/protein of interest has been secreted into the culture medium. The immunocytochemical technique illustrated in the Examples may also be used.

Lipid globule targeting sequences used according to the present invention are also preferably capable of displacing ADRP from intracellular lipid globules and/or of reducing the levels of ADRP protein in a cell when expressed in, or administered to, the cell (as illustrated for HCV core protein in the Examples). Suitable techniques for determining whether a candidate sequence has these properties are described below.

Suitable lipid globule targeting sequences may be obtained from an HCV core protein. The amino acid sequence of the HCV core protein has been obtained for a large number of different HCV isolates. These sequences are readily available to the skilled person. One such sequence, for HCV strain Glasgow, is set out in SEQ ID No. 1. The means for cloning and identifying new HCV strains, and thus obtaining further core sequences, are described in EP-B-318,216.

According to the present invention, it is preferred to use fragments of the HCV core protein which are capable of targeting molecules, to which they are linked, to lipid globules. Amino acid numbering for preferred fragments set out below is with reference to SEQ ID. No. 1. However it will be understood that equivalent fragments of the core protein of other HCV strains/isolates may also be used. An HCV core protein-derivable lipid globule targeting sequence of the invention is preferably a minimal amino acid sequence which can target a molecule, typically a protein, to lipid globules. The minimal sequence will typically comprise a hydrophobic amino acid sequence derived from amino acids 120 to 169 of an HCV core sequence, preferably linked to a hydrophilic amino acid sequence of at least 8, preferably 10, more preferably at least 12 amino acids. It is not necessary for the hydrophilic sequence to be contiguous with the hydrophobic sequence. For example, a protein of interest may be placed between the two sequences such that the hydrophilic sequence is at the N-terminus and the hydrophobic sequence is at the C-terminus.

The hydrophobic amino acid sequence typically comprises at least 10, preferably at least 15 or 20 contiguous amino acids and has a hydropathy index of at least +40 kJ/mol (determined, for example, theoretically as described by Engelman et al., 1986. The hydrophilic amino acid sequence typically has a hydropathy plot of less than −20 kJ/mol, preferably less than −40 kJ/mol.

Preferred HCV core fragments contain amino acids 161 to 166 (SEQ ID. No. 3). It is also preferred to use fragments of the HCV core protein that contain amino acids 125 to 144 (SEQ ID. No. 2). In a preferred embodiment, HCV core protein fragments of the invention contain both amino acids 125 to 144 and amino acids 161 to 166. In an especially preferred embodiment, the lipid targeting sequence of the invention comprises a hydrophilic amino acid sequence containing amino acids 1 to 8 of the HCV core sequence. Other preferred fragments contain amino acids 1 to 173 or 1 to 169.

Since it has also now been shown that amino acids 9 to 43, 49 to 75, 80 to 118 and 155 to 161 are not required for lipid association, preferred HCV core protein fragments of the invention lack one or more of these sequences. Suitable fragments will be at least about 5, e.g. 10, 12, 15 or 20 amino acids in size and preferably have less than 100, 90, 80, 70, 60 or 50 amino acids. In a preferred aspect, fragments contain an HCV epitope.

Lipid globule targeting sequences of the invention, for example HCV core protein sequences and fragments thereof, may, however, be part of a larger polypeptide, for example a fusion protein. In this case, the additional polypeptide sequences are preferably polypeptide sequences with which the lipid globule targeting sequence of the invention is not normally associated.

It will be understood that lipid globule targeting sequences of the invention are not limited to sequences obtained from HCV core protein but also include homologous sequences obtained from any source, for example related viral proteins, cellular homologues and synthetic peptides, as well as variants or derivatives thereof. Thus, the present invention covers variants, homologues or derivatives of the targeting sequences of the present invention, as well as variants, homologues or derivatives of the nucleotide sequence coding for the targeting sequences of the present invention.

In the context of the present invention, a homologous sequence is taken to include an amino acid sequence which is at least 60, 70, 80 or 90% identical, preferably at least 95 or 98% identical at the amino acid level over at least 5, preferably 8, 10, 15, 20, 30 or 40 amino acids with an HCV core protein lipid targeting sequence, for example as shown in the sequence listing herein. In particular, homology should typically be considered with respect to those regions of the targeting sequence known to be essential for lipid globule association rather than non-essential neighboring sequences. Homology comparisons can be conducted by eye, or more usually, with the aid of readily available sequence comparison programs. These commercially available computer programs can calculate % homology between two or more sequences. A typical example of such a computer program is CLUSTAL.

Sequence homology (or identity) may moreover be determined using any suitable homology algorithm, using for example default parameters. Advantageously, the BLAST algorithm is employed, with parameters set to default values. The BLAST algorithm is described in detail at http://www.ncbi.nih.gov/BLAST/blast_help.html, which is incorporated herein by reference. The search parameters are defined as follows, and are advantageously set to the defined default parameters.

Advantageously, “substantial homology” when assessed by BLAST equates to sequences which match with an EXPECT value of at least about 7, preferably at least about 9 and most preferably 10 or more. The default threshold for EXPECT in BLAST searching is usually 10.

BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul (see http://www.ncbi.nih.gov/BLAST/blast help.html) with a few enhancements. The BLAST programs were tailored for sequence similarity searching, for example to identify homologues to a query sequence. The programs are not generally useful for motif-style searching. For a discussion of basic issues in similarity searching of sequence databases, see Altschul et al., 1994.

The five BLAST programs available at http://www.ncbi.nlm.nih.gov perform the following tasks:

blastp—compares an amino acid query sequence against a protein sequence database;

blastn—compares a nucleotide query sequence against a nucleotide sequence database;

blastx—compares the six-frame conceptual translation products of a nucleotide query sequence (both strands) against a protein sequence database;

tblastn—compares a protein query sequence against a nucleotide sequence database dynamically translated in all six reading frames (both strands).

tblastx—compares the six-frame translations of a nucleotide query sequence against the six-frame translations of a nucleotide sequence database.

BLAST uses the following search parameters:

HISTOGRAM—Display a histogram of scores for each search; default is yes. (See parameter H in the BLAST Manual).

DESCRIPTIONS—Restricts the number of short descriptions of matching sequences reported to the number specified; default limit is 100 descriptions. (See parameter V in the manual page). See also EXPECT and CUTOFF.

ALIGNMENTS—Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

EXPECT—The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF—Cutoff score for reporting high-scoring segment pairs (HSPs). The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

MATRIX—Specify an alternate scoring matrix for BLASTP, BLASTX, TBLASTN and TBLASTX. The default matrix is BLOSUM62 (Henikoff & Henikoff, 1992). The valid alternative choices include: PAM40, PAM120, PAM250 and IDENTITY. No alternate scoring matrices are available for BLASTN; specifying the MATRIX directive in BLASTN requests returns an error response.

STRAND—Restrict a TBLASTN search to just the top or bottom strand of the database sequences; or restrict a BLASTN, BLASTX or TBLASTX search to just reading frames on the top or bottom strand of the query sequence.

FILTER—Mask off segments of the query sequence that have low compositional complexity, as determined by the SEG program of Wootton & Federhen (1993), or segments consisting of short-periodicity internal repeats, as determined by the XNU program of Clayerie & States (1993), or, for BLASTN, by the DUST program of Tatusov and Lipman (see http://www.ncbi.nlm.nih.gov). Filtering can eliminate statistically significant but biologically uninteresting reports from the blast output (e.g. hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “NNNNNNNNNNNNN”) and the letter “X” in protein sequences (e.g., “XXXXXXXXX”).

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi Causes NCBI gi identifiers to be shown in the output, in addition to the accession and/or locus name.

Most preferably, sequence comparisons are conducted using the simple BLAST search algorithm provided at http://www.ncbi.nlm.nih.gov/BLAST.

Other computer program methods to determine identify and similarity between the two sequences include but are not limited to the GCG program package (Devereux et al 1984) and FASTA (Altschul et al. 1990).

Lipid globule targeting sequences of the invention, for example HCV core protein sequences, variants, homologues and fragments thereof, may be modified for use in the present invention. Typically, modifications are made that maintain the hydrophobicity/hydrophilicity of the sequence Amino acid substitutions may be made, for example from 1, 2 or 3 to 10, 20 or 30 substitutions provided that the modified sequence retains the ability to target molecules to lipid globules. Amino acid substitutions may include the use of non-naturally occurring analogues, for example to increase blood plasma half-life of a therapeutically administered polypeptide.

Conservative substitutions may be made, for example according to the Table below. Amino acids in the same block in the second colunm and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The terms “variant”, “homologue” or “derivative” in relation to the targeting sequence of the present invention include any substitution of, variation of, modification of, replacement of, deletion of or addition of one (or more) amino acids from or to the sequence providing the resultant amino acid sequence has a lipid globule targeting activity, preferably having at least the same activity of the targeting sequences presented in the sequence listings.

Proteins of the invention comprising lipid globule targeting sequences are typically made by recombinant means, for example as described below. However they may also be made by synthetic means using techniques well known to skilled persons such as solid phase synthesis. Proteins of the invention may also be produced as fusion proteins, for example to aid in extraction and purification. Examples of fusion protein partners include glutathione-S-transferase (GST), 6×His, GAL4 (DNA binding and/or transcriptional activation domains) and β-galactosidase. It may also be convenient to include a proteolytic cleavage site between the fusion protein partner and the HCV core protein sequence and/or between the HCV core protein sequence and the protein of interest to allow removal of fusion protein sequences. Preferably the fusion protein will not hinder the lipid targeting effect of the lipid globule targeting sequence. The targeting sequence may be linked to either the N-terminus or the C-terminus of the fusion protein partners or proteins of interest

Proteins of the invention may be in a substantially isolated form. It will be understood that the protein may be mixed with carriers or diluents which will not interfere with the intended purpose of the protein and still be regarded as substantially isolated. A protein of the invention may also be in a substantially purified form, in which case it will generally comprise the protein in a preparation in which more than 90%, e.g. 95%, 98% or 99% of the protein in the preparation is a protein of the invention.

2. Adipocyte-specific Differentiation Related Protein

In another embodiment of the in vitro assay methods of the present invention, ADRP may be added to the mixture to compete with a lipid globule targeting sequence. ADRP protein (also known as adipophilin) can be obtained in a number of ways, for example by purification from mammalian milk or mammalian cell lines (Heid et al., 1998). Alternatively, ADRP can be obtained by recombinant means, as described above for lipid globule targeting sequences. The nucleotide sequence of human ADRP, for example, is given in U.S. Pat. No. 5,739,009 and shown in SEQ ID. No. 4. The polypeptide sequence of human ADRP is shown in SEQ ID No. 5. Since ADRP is capable of binding lipid globules, it can also be considered to be a lipid globule targeting sequence within the scope of the invention. Consequently, the disclosure above and below relating to lipid globule targeting sequences also generally applies, where appropriate, to ADRP sequences, for example the use of fragments, homologues, derivatives and variants. In particular, ADRP sequences may be modified for use in the assays of the present invention as described above.

B. Polynucleotides and Vectors.

Polynucleotides of the invention comprise nucleic acid sequences encoding the lipid globule targeting sequences of the invention. It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

Polynucleotides of the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Such modifications may be carried out in order to enhance the in vivo activity or life span of polynucleotides of the invention.

The terms “variant”, “homologue” or “derivative” in relation to the nucleotide sequence coding for the lipid targeting sequence of the present invention include any substitution of, variation of, modification of, replacement of, deletion of or addition of one (or more) nucleic acid from or to the sequence providing the resultant nucleotide sequence codes for a protein having lipid targeting activity, preferably having at least the same activity of the targeting sequences presented in the sequence listings.

As indicated above, with respect to sequence homology, preferably there is at least 75%, more preferably at least 85%, more preferably at least 90% homology to the sequences shown in the sequence listing herein. More preferably there is at least 95%, more preferably at least 98%, homology. Nucleotide homology comparisons may be conducted as described above.

The present invention also encompasses nucleotide sequences that are capable of hybridizing selectively to the sequences presented herein, or any variant, fragment or derivative thereof, or to the complement of any of the above. Nucleotide sequences are preferably at least 15 nucleotides in length, more preferably at least 20, 30, 40 or 50 nucleotides in length.

The term “hybridization” as used herein shall include “the process by which a strand of nucleic acid joins with a complementary strand through base pairing” (Coombs J (1994) Dictionary of Biotechnology, Stockton Press, New York N.Y.) as well as the process of amplification as carried out in polymerase chain reaction technologies as described in Dieffenbach C W and G S Dveksler (1995, PCR Primer, a Laboratory Manual, Cold Spring Harbor Press, Plainview N.Y.).

Polynucleotides of the invention capable of selectively hybridizing to the nucleotide sequences presented herein, or to their complement, will be generally at least 70%, preferably at least 80 or 90% and more preferably at least 95% or 98% homologous to the corresponding nucleotide sequences presented herein over a region of at least 20, preferably at least 25 or 30, for instance at least 40, 60 or 100 or more contiguous nucleotides. Preferred polynucleotides of the invention will comprise regions homologous to nucleotides 715 to 774 and/or nucleotides 826 to 840 of SEQ ID No. 1, preferably at least 80 or 90% and more preferably at least 95% homologous to nucleotides 715 to 774 and/or nucleotides 826 to 840 of SEQ ID No. 1.

The term “selectively hybridizable” means that the polynucleotide used as a probe is used under conditions where a target polynucleotide of the invention is found to hybridize to the probe at a level significantly above background. The background hybridization may occur because of other polynucleotides present, for example, in the cDNA or genomic DNA library being screening. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabeling the probe, e.g. with ³²P.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical polynucleotide sequences while an intermediate (or low) stringency hybridization can be used to identify or detect similar or related polynucleotide sequences.

In a preferred aspect, the present invention covers nucleotide sequences that can hybridize to the nucleotide sequence of the present invention under stringent conditions (e.g. 65° C. and 0.1×SSC{1×SSC=0.15 M NaCl, 0.015 M Na₃ citrate pH 7.0).

Where the polynucleotide of the invention is double-stranded, both strands of the duplex, either individually or in combination, are encompassed by the present invention. Where the polynucleotide is single-stranded, it is to be understood that the complementary sequence of that polynucleotide is also included within the scope of the present invention.

Polynucleotides which are not 100% homologous to the sequences of the present invention but fall within the scope of the invention can be obtained in a number of ways. Other HCV core protein variants of the HCV core protein sequence described herein may be obtained for example by probing DNA libraries made from a range of HCV infected individuals, for example individuals from different populations. In addition, other viral, or cellular homologues particularly cellular homologues found in mammalian cells (e.g. rat, mouse, bovine and primate cells), may be obtained and such homologues and fragments thereof in general will be capable of selectively hybridizing to the sequences shown in the sequence listing herein. Such sequences may be obtained by probing cDNA libraries made from or genomic DNA libraries from other animal species, and probing such libraries with probes comprising all or part of SEQ ID. 1 under conditions of medium to high stringency. Similar considerations apply to obtaining species homologues and allelic variants of ADRP.

Variants and strain/species homologues may also be obtained using degenerate PCR which will use primers designed to target sequences within the variants and homologues encoding conserved amino acid sequences within the lipid globule targeting sequences of the present invention. Conserved sequences can be predicted, for example, by aligning the HCV core protein amino acid sequences from several HCV isolates. Such HCV sequence comparisons are widely available in the art. The primers will contain one or more degenerate positions and will be used at stringency conditions lower than those used for cloning sequences with single sequence primers against known sequences.

Alternatively, such polynucleotides may be obtained by site directed mutagenesis of characterized lipid globule targeting sequences, such as SEQ ID. No 1. This may be useful where for example silent codon changes are required to sequences to optimize codon preferences for a particular host cell in which the polynucleotide sequences are being expressed. Other sequence changes may be desired in order to introduce restriction enzyme recognition sites, or to alter the property or function of the polypeptides encoded by the polynucleotides.

Polynucleotides of the invention may be used to produce a primer, e.g. a PCR primer, a primer for an alternative amplification reaction, a probe e.g. labeled with a revealing label by conventional means using radioactive or non-radioactive labels, or the polynucleotides may be cloned into vectors. Such primers, probes and other fragments will be at least 15, preferably at least 20, for example at least 25, 30 or 40 nucleotides in length, and are also encompassed by the term polynucleotides of the invention as used herein.

Polynucleotides such as a DNA polynucleotides and probes according to the invention may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer polynucleotides will generally be produced using recombinant means, for example using a PCR polymerase chain reaction) cloning techniques. This will involve making a pair of primers (e.g. of about 15 to 30 nucleotides) flanking a region of the lipid targeting sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector

Polynucleotides of the invention can be incorporated into a recombinant replicable vector. The vector may be used to replicate the nucleic acid in a compatible host cell. Thus in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells include bacteria such as E. coli, yeast, mammalian cell lines and other eukaryotic cell lines, for example insect Sf9 cells.

Preferably, a polynucleotide of the invention in a vector is operably linked to a control sequence that is capable of providing for the expression of the coding sequence by the host cell, i.e. the vector is an expression vector. The term “operably linked” means that the components described are in a relationship permitting them to function in their intended manner. A regulatory sequence “operably linked” to a coding sequence is ligated in such a way that expression of the coding sequence is achieved under condition compatible with the control sequences.

A particularly preferred vector for use in the present invention comprises the control sequences naturally associated with ADRP genes, such as the human ADRP gene. Typically, the control sequences are operably linked to a reporter gene. Such a vector may be used in the assays described below for identifying modulators of ADRP expression. The control sequences may be modified, for example by the addition of further transcriptional regulatory elements to make the level of transcription directed by the control sequences more responsive to transcriptional modulators.

Vectors of the invention may be transformed or transfected into a suitable host cell as described below to provide for expression of a protein of the invention. This process may comprise culturing a host cell transformed with an expression vector as described above under conditions to provide for expression by the vector of a coding sequence encoding the protein, and optionally recovering the expressed protein. Alternatively, vectors comprising the control sequences naturally associated with ADRP genes operably linked to a reporter gene as a reporter construct may be transformed or transfected into a host cell, for example for use in assays of the invention which measure the effect of candidate substances on transcription from the reporter construct in a cellular environment.

The vectors may be for example, plasmid or virus vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. Vectors may be used, for example, to transfect or transform a host cell.

Control sequences operably linked to sequences encoding the protein of the invention include promoters/enhancers and other expression regulation signals. These control sequences may be selected to be compatible with the host cell for which the expression vector is designed to be used in. The term promoter is well-known in the art and encompasses nucleic acid regions ranging in size and complexity from minimal promoters to promoters including upstream elements and enhancers.

The promoter is typically selected from promoters which are functional in mammalian, cells, although prokaryotic promoters and promoters functional in other eukaryotic cells may be used. The promoter is typically derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of α-actin, β-actin, tubulin) or, alternatively, a tissue-specific manner (such as promoters of the genes for pyruvate kinase). Tissue-specific promoters specific for liver cells are particularly preferred, for example hepatitis B viral promoters, apoliprotein AII promoters human serum amyloid P component promoters or human protein C gene promoters. They may also be promoters that respond to specific stimuli, for example promoters that bind steroid hormone receptors. Viral promoters may also be used, for example the Moloney murine leukemia virus long terminal repeat (MMLV LTR) promoter, the rous sarcoma virus (RSV) LTR promoter or the human cytomegalovirus (CMV) IE promoter. As discussed above ADRP promoters are particularly preferred for use in reporter constructs.

It may also be advantageous for the promoters to be inducible so that the levels of expression of the heterologous gene can be regulated during the life-time of the cell. Inducible means that the levels of expression obtained using the promoter can be regulated.

In addition, any of these promoters may be modified by the addition of further regulatory sequences, for example enhancer sequences. Chimeric promoters may also be used comprising sequence elements from two or more different promoters described above.

C. Host cells

Vectors and polynucleotides of the invention (and reporter gene constructs described below) may be introduced into host cells for the purpose of replicating the vectors/polynucleotides and/or expressing the proteins of the invention encoded by the polynucleotides of the invention. Although the proteins of the invention may be produced using prokaryotic cells as host cells, it is preferred to use eukaryotic cells, for example yeast, insect or mammalian cells, in particular mammalian cells. Particularly preferred cells are those with substantial amounts of intracellular lipid droplets/globules, for example adipocytes. Cells which are targeted by a particular virus which it is desired to treat, for example liver cells, are also preferred.

Vectors/polynucleotides of the invention may introduced into suitable host cells using a variety of techniques known in the art, such as transfection, transformation and electroporation. Where vectors/polynucleotides of the invention are to be administered to animals, several techniques are known in the art, for example infection with recombinant viral vectors such as herpes simplex viruses and adenoviruses, direct injection of nucleic acids and biolistic transformation.

D. Protein Expression and Purification

Host cells comprising polynucleotides of the invention may be used to express proteins of the invention. Host cells may be cultured under suitable conditions which allow expression of the proteins of the invention. Expression of the proteins of the invention may be constitutive such that they are continually produced, or inducible, requiring a stimulus to initiate expression. In the case of inducible expression, protein production can be initiated when required by, for example, addition of an inducer substance to the culture medium, for example dexamethasone or IPTG.

Proteins of the invention can be extracted from host cells by a variety of techniques known in the art, including enzymatic, chemical and/or osmotic lysis and physical disruption. Although a large number of different purification protocols may be used, given the ability of the HCV core proteins of the invention to target proteins of interest to lipid globules, a preferred extraction/purification protocol involves centrifuging cell homogenates at high speed (for example 100,000 g for 60 mins at 2 to 4° C.) and removing the resulting layer of floating lipids. This will function as a primary purification step. Further purification can then be performed if necessary using, for example, column chromatography such as ion-exchange or affinity chromatography. Cells which secrete lipid globules may also conveniently be used and the lipid globules harvested from the culture supernatant.

Proteins associated with the membrane surrounding fat globules can be fractionated into soluble and insoluble fractions by extraction with 1% (w/v) Triton X-100/1.5 M NaCl/10 mM Tris (pH 7.0), by extraction with 1.5% (w/v) dodecyl β-D maltoside/0.75 M aminohexanoic acid/10 mM Hepes (pH 7.0) or by sequential extraction with these two detergent-containing solutions (Patton and Huston, 1986). Suspension of the fat globule components in the detergent-containing solution can be achieved by using an all-glass homogenizer, and keeping on ice for 30 to 60 min, after which insoluble and soluble materials can be separated by centrifugation for 60 min at 2° C. and 150,000 g. The above conditions can be modified to analyze whether core protein or a fusion protein containing core as a component is attached to fat globules. Other detergents, both ionic and non-ionic, along with salt solutions at various concentrations could be used to derive the proteinaceous material from fat globules. The incubation times and temperatures may be optimized by empirical means.

E. Assays

1. Assays for Substances that Disrupt the Interaction Between a Lipid Globule Targeting Sequence and a Lipid Globule

i. Candidate Substances

A substance which disrupts an interaction between the lipid globule targeting sequence and lipid globule may do so in several ways. It may directly disrupt the binding of the lipid globule targeting sequence to the lipid globule by, for example, binding to the lipid globule targeting sequence and masking or altering the site of interaction with the lipid globule. Alternatively, the candidate substance may compete for binding sites on the lipid globule surface and displace the lipid globule targeting sequence. Candidate substances of these types may conveniently be screened by in vitro binding assays as, for example, described below. Candidate substances may also be screened using an “in vivo” whole cell assay as described below. The term ‘in vivo’ is intended to encompass experiments with cells in culture as well as experiments with intact multicellular organisms.

A substance which can bind directly to the lipid globule targeting sequence may also inhibit an interaction between the lipid globule targeting sequence and the lipid globule by altering the subcellular localization of the lipid globule targeting sequence thus preventing the two components from coming into contact within the cell. This can also be tested in vivo using, for example the in vivo assays described below.

Alternatively, instead of preventing the association of the components directly, the substance may suppress or enhance the biologically available amount of lipid globule targeting sequence (for example a viral protein component). This may be by inhibiting expression of the viral protein comprising the lipid globule targeting sequence, for example at the level of transcription, transcript stability, translation, post-translational processing or post-translational stability. An example of such a substance would be antisense RNA which suppresses the amount of HCV core protein mRNA translated into protein.

Suitable candidate substances include viral peptides, which in some cases may especially be of from about 5 to 20 amino acids in size, based on, for example, lipid globule targeting motifs found within the HCV core protein, or variants of such peptides in which one or more residues have been substituted. Peptide fragments of ADRP may also be used, including modified variants thereof. Peptides from panels of peptides comprising random sequences or sequences which have been varied consistently to provide a maximally diverse panel of peptides may be used.

Suitable candidate substances also include antibody products (for example, monoclonal and polyclonal antibodies, single chain antibodies, chimeric antibodies and CDR-grafted antibodies) which are specific for the lipid globule targeting sequence or surface constituents of the lipid globules. Furthermore, combinatorial libraries, peptide and peptide mimetics, defined chemical entities such as organic and inorganic compounds, oligonucleotides, and natural product libraries may be screened for activity as inhibitors of an interaction between the lipid globule targeting sequence and a lipid globule in assays such as those described below. The candidate substances may be used in an initial screen in batches of, for example 10 substances per reaction, and the substances of those batches which show inhibition tested individually. Candidate substances which show activity in in vitro screens such as those described below can then be tested in in vivo systems, such as mammalian cells which will be exposed to the inhibitor and tested, for example, for susceptibility to viral infection.

ii. Assays

The assays of the invention may be in vitro assays or in vivo assays, for example using cell lines or an animal model, such as mice or chimpanzees.

In vitro Assay Systems

One type of in vitro assay for identifying substances which disrupt an interaction between the lipid globule targeting sequence and a lipid globule involves measuring in the presence or absence of a candidate substance the association of a targeting sequence with lipid globules formed in vitro. Binding of targeting sequences to lipid globules may be assessed by adding a targeting sequence to a dispersion of lipid globules (such as a mixture of phospholipid and triacylglycerol) in an aqueous solvent, sonicating the mixture and determining by fractionation the degree of partition between aqueous and lipid phases. Typically fractionation of the mixture would involve increasing the density of the solution with sorbitol or sodium bromide and ultracentrifuging the solution. The lipid complexes migrate to the top of the centrifuge tube and this upper lipid layer is then examined for targeting sequence. This is performed in the presence or absence of the candidate substance whose inhibitory activity it is desired to test. Typically, a candidate substance is deemed to inhibit the lipid globule targeting sequence if it causes a reduction of at least 50%, preferably at least 60, 70, 80 or 90% in the amount of targeting sequence associated with the lipid phase in the absence of the candidate substance.

The candidate substance may be pre-incubated with the lipid globule targeting sequence or with a lipid globule or added to the reaction mixture after pre-incubation of the lipid globule targeting sequence with a lipid globule.

Another type of in vitro assay for identifying substances which disrupt an interaction between the lipid globule targeting sequence and a lipid globule or a lipophilic surface involves the following:

Proteins incorporating the lipid globule targeting sequence, for example a fragment of HCV core protein, and optionally ADRP, may be translated in vitro from RNA transcripts encoding these polypeptides. Reactions would be supplemented with membranes originating from tissue culture cells (e.g. microsomal membranes) or a lipophilic surface which had been artificially created (e.g. a liposome) to which the in vitro translated proteins can bind. Binding may be assessed by purification of the lipid components of reactions (e.g. by centrifugation). Alternatively a chip format (for example BIAcore™—Biacore AB) wherein the chip has a lipid surface may be used to assess binding.

The ability of candidate substances to disrupt association of the targeting sequence and/or ADRP with lipid can be examined by adding to reactions candidate substances during synthesis of the proteins. Thus, the effects of substances on core/lipid globule targeting sequences and ADRP association with lipid can be compared. Preferably, a suitable inhibitor of a lipid globule targeting sequence is capable of inhibiting binding of viral lipid targeting sequences such as HCV core to lipid globules without affecting binding of ADRP.

In vivo Assay Systems

In vivo assays for identifying compounds that disrupt an interaction between the lipid globule targeting sequence and a lipid globule typically involve administering a candidate substance to a cell which expresses a lipid globule targeting sequence, for example a fragment of HCV core protein, and determining if the ability of the targeting sequence to associate with intracellular lipid globules has been affected, for example reduced or abolished. Thus the association of the targeting sequence with intracellular lipid globules is determined in the absence of the candidate substance and in the presence of the candidate substance and the results compared.

Association of the lipid globule targeting sequence with intracellular globules is typically determined by immunofluorescence microscopy using an antibody which recognizes the targeting sequence and an antibody or stain which recognizes intracellular lipid globules or a surface component of the globules. If the targeting sequences is able to bind to the lipid globules, then the targeting sequence will substantially co-localize with the lipid globules. A suitable procedure is described in the Examples. In addition, since we show that HCV core protein can displace ADRP from lipid globules, the co-localization of endogenous ADRP with lipid globules may also be determined in the presence and absence of the candidate substance.

A candidate substance is generally considered to be capable of disrupting the interaction between the lipid globule targeting sequence and intracellular lipid globules if, as determined by immunofluorescence microscopy, less than 50% of the detectable targeting sequence protein co-localizes with intracellular lipid globules, preferably less than 60%, more preferably less than 70, 80 or 90%. Preferably, a suitable inhibitor of a lipid globule targeting sequence is capable of disrupting the interaction of viral lipid targeting sequences such as HCV core with intracellular lipid globules/surface components without affecting the interaction of ADRP with intracellular lipid globules/surface components.

It will be appreciated by the skilled person that other techniques are available for determining localization of proteins and lipid within intact cells and that these techniques are also applicable to the assays of the present invention.

The candidate substance, i.e. the test compound, may be administered to the cell in one or more ways. For example, it may be added directly to the cell culture medium or injected into the cell. Alternatively, in the case of polypeptide candidate substances, the cell may be transfected with a nucleic acid construct which directs expression of the polypeptide in the cell. Preferably, the expression of the polypeptide is under the control of a regulatable promoter, for example so that expression is induced shortly before it is desired to administer the candidate substance.

Another suitable test may involve introducing a polynucleotide encoding a targeting sequence of the invention, optionally linked to a protein of interest, into a milk-producing cell in culture and determining whether, the targeting sequence/protein of interest has been secreted into the culture medium. This would be carried out in the presence or absence of the candidate substance.

2. Assays for Substances Capable of Modulating ADRP Expression in Cells

i. Candidate Substances

Substances that modulate ADRP expression, preferably up-regulate ADRP expression so that the levels of ADRP protein in a cell are increased, may do so by one or more of several mechanisms. For example, a substance may increase levels of transcription from endogenous ADRP genes, stabilizes ADRP mRNA levels and/or stabilizes ADRP protein. Transcription may be increased from endogenous ADRP genes by, for example, the use of a transcriptional activator that activates transcription from endogenous ADRP genes or a substance that modifies the effect of a transcriptional inhibitor of endogenous ADRP genes. Thus tests for modulation of ADRP expression include determining the effect of a candidate substance on ADRP mRNA levels and/or ADRP protein levels.

The term “modulate” in the context of the ADRP expression means a change or alteration in the levels of ADRP mRNA and/or ADRP protein.

Suitable candidate substances include substances known to modulate cellular transcription and/or lipid metabolism. Examples of substances which may affect ADRP expression include non-steroidal anti-inflammatory drugs such as ibuprofen and indomethacin, and drugs known to affect lipid metabolism such as fibrates and thiazolidinediones. Furthermore, combinatorial libraries, peptide and peptide mimetics, in particular peptides from panels of peptides comprising random sequences or sequences which have been varied consistently to provide a maximally diverse panel of peptides, defined chemical entities such as organic and inorganic compounds, oligonucleotides, and natural product libraries may be screened for activity as modulators of ADRP expression in assays such as those described below. The candidate substances may be used in an initial screen in batches of, for example 10 substances per reaction, and the substances of those batches which show inhibition tested individually. In addition, candidate substances which show activity in in vitro screens such as those described below can then be tested in in vivo systems, such as mammalian cells which will be exposed to the inhibitor and tested, for example, for susceptibility to viral infection.

ii. Assays

The assays of the invention may be in vitro assays or in vivo assays, for example using cell lines or an animal model.

In vitro Assay Systems

An in vitro assay system of the invention typically measures the effect on transcription from a polynucleotide construct comprising an ADRP promoter linked to a polynucleotide whose transcribed, and optionally translated, product is capable of detection, for example the naturally occurring ADRP coding sequence or a reporter gene such as luciferase. Techniques for detecting and quantitating transcription products are well known in the art and include, for example hybridisation to labeled probes and direct quantitation of transcribed products by the use of labeled nucleotides which are incorporated during transcription. Translated products may also be detected using well known techniques such as SDS-PAGE and Western blotting, or in the case of biologically active products, suitable assays for detecting said activity (such as CAT assays or chemiluminescence assays).

A suitable in vitro assay may for example, be conducted using extracts of mammalian cells, typically supplemented with buffers and nucleotide mixes. Reporter constructs comprising polynucleotides containing ADRP promoter constructs linked to a reporter gene may be added to the mix, or endogenous genomic DNA used.

The effect of a candidate substance on ADRP expression may be determined by measuring levels of transcription from the ADRP promoter construct (endogenous or otherwise) in the presence and absence of the candidate substance and comparing the results. Preferably, a control promoter construct should also be tested to ensure that any effect on ADRP expression is specific to the ADRP promoter and is not simply the result of general transcriptional inhibition. A candidate substance is typically considered to modulate ADRP expression if the levels of transcriptional are altered by at least 30%, preferably at least 50, 60, 70, 80 or 90%. Any affect on the control promoter should preferably be accounted for when calculating changes in ADRP transcription.

In vivo Assay Systems

Modulation of ADRP expression can also conveniently be measured in vivo, typically using mammalian cell lines. As with in vitro systems, both reporter constructs and endogenous ADRP genes may be used. In a preferred embodiment, the assay uses mammalian cells stably transfected with a polynucleotide comprising a reporter construct which contains an ADRP promoter operably linked to a reporter gene, for example chloramphenicol transferase (CAT) or luciferase. The cell also preferably comprises a stably transfected control promoter sequence operably linked to a second reporter gene which can be distinguished from the first reporter gene. Typically, levels of transcription from the ADRP construct and the control construct are measured in the absence of a candidate substance and then measured in the presence of a candidate substance. The effect of the candidate substance on transcription from the ADRP reporter construct (or endogenous ADRP gene) can then be determined, taking into account any general effect on transcription as indicated by the result obtained for the control reporter construct.

In another embodiment, ADRP expression is measured by determining the amount of ADRP protein in cells before administration of the candidate substance and after administration of the candidate substance. As described above, protein levels are typically measured by analysing cell extracts by SDS-PAGE and detecting the ADRP protein using Western blotting. Alternatively, the cells used in the assay of the invention may comprise a reporter construct containing an ADRP promoter operably linked to a nucleotide sequence encoding a detectable polypeptide product, such as CAT. In a particularly preferred embodiment, the detectable product encodes an enzyme which can cleave a cellular or exogenously added substance causing a detectable change in the absorption spectrum or emission spectrum of the cell or cell medium at a particular wavelength. This will facilitate the large-scale screening of candidate substances in, for example a microtiter plate assay format.

Administration of candidate substances to mammalian cell lines and generation of host mammalian cell lines comprising reporter constructs can be carried out as described above.

3. Testing Candidate Substances for Anti-viral Activity

Candidate substances that are identified by the method of the invention as disrupting an interaction between a lipid globule targeting sequence and a lipid globule, or modulating ADRP expression may be tested for their ability to, for example, reduce susceptibility of cells to viral infection. Such compounds could be used therapeutically to affect viral infection, for example to prevent or treat viral infection.

Typically, an assay to determine the effect of a candidate substance identified by a method of the invention on the susceptibility of cells to viral infection comprises:

(a) administering a virus, for example HCV, to a cell, in the absence of the candidate substance;

(b) administering the virus to the cell in the presence of the candidate substance; and

(c) determining if the candidate substance reduces or abolishes the susceptibility of the cell to viral infection.

The candidate substance may be administered before, or concomitant with, the virus to establish if infection is prevented. Alternatively, the candidate substance may be administered subsequent to viral infection to establish if viral infection can be treated using the candidate substance. Administration of candidate substances to cells may be performed as described above.

The assay is typically carried out using mammalian cell lines, but an animal model could be employed instead, such as chimpanzees in the case of HCV. The virus is contacted with cells, typically cells in culture. The cells may be cells of a mammalian cell line, in particular mammalian cells susceptible to infection by the virus in the absence of the candidate substance, for example in the case of HCV, liver cells.

Techniques for assaying infectivity of viruses are well-known in the art. As well as using plaque assays, levels of viral infection can be determined by using recombinant viruses which comprise a reporter gene, for example lacZ. The use of a histochemically detectable reporter gene is especially preferred when experiments are performed with animals. In the case of HCV, plaque assays cannot be used. A suitable method for determining the level of HCV production in an infected animal is to perform RT-PCR analysis of the amount of positive-strand nucleic acid material in cells or serum. In addition, the presence of negative-strand RNA in cells, as determined by RT-PCR, is taken to mean that there is active viral replication.

In a preferred embodiment of the above-described assays, lipid globule targeting sequence and derivatives thereof are used in an experimental system to study normal cellular interactions. For example, derivatives of viral proteins such as HCV core protein or derivatives of ADRP, including deletion, insertion and substitution mutants, can be used to disrupt an interaction between ADRP and lipid globules. This can be tested in vitro or in vivo using the assays described above. The interaction between ADRP and lipid globules can also be disrupted in vivo by introducing a lipid globule targeting sequence or derivatives thereof, including deletion, insertion and substitution mutants, into cells in vivo, preferably mammalian cells, more preferably human cells.

Lipid globule targeting sequences and their derivatives can be introduced into the cells using techniques described above, for example transfection of nucleic acid constructs encoding lipid globule targeting sequences, or using viral vectors. The effect of this disruption can be determined as described above. Any in vitro data obtained may be used to assist in the rational design of lipid globule targeting sequences for use in the in vivo studies. In addition, the precise regions/amino acid residues of lipid globule targeting sequences which bind to lipid globules can determined by in vitro binding studies using lipid globule targeting sequence derivatives. This will also assist in the rational design of lipid globule targeting sequence derivatives for use in the in vivo studies.

Thus viral lipid globule targeting sequences, which are readily distinguished from cellular constituents, may be used as a tool to investigate intracellular lipid metabolism.

F. Therapeutic Uses

Substances capable of disrupting an interaction between a viral lipid globule targeting sequence and an intracellular globule may be used to affect a viral infection in a human or animal, in particular to treat or prevent a viral infection. Such substances may have been identified by the assay methods of the invention or otherwise.

Substances that are capable of modulating ADRP expression may also be used to affect a viral infection in a human or animal, in particular to treat or prevent a viral infection. Such substances may have been identified by the assay methods of the invention or otherwise. For example, the non-steroidal anti-inflammatory drugs ibuprofen and indomethacin have been shown to stimulate ADRP expression. Consequently, the present invention provides the use of ibuprofen and/or indomethacin in the manufacture of a medicament for use in affecting a viral infection, preferably an HCV infection.

G. Compositions/Administration

Proteins of the invention and substances identified or identifiable by the assay methods of the invention may preferably be combined with various components to produce compositions of the invention. Preferably the compositions are combined with a pharmaceutically acceptable carrier or diluent to produce a pharmaceutical composition (which may be for human or animal use). Suitable carriers and diluents include isotonic saline solutions, for example phosphate-buffered saline. The composition of the invention may be administered by direct injection. The composition may be formulated for parenteral, intramuscular, intravenous, subcutaneous, intraocular or transdermal administration. Typically, each protein may be administered at a dose of from 0.01 to 30 mg/kg body weight, preferably from 0.1 to 10 mg/kg, more preferably from 0.1 to 1 mg/kg body weight.

Polynucleotides/vectors encoding polypeptide components for use in affecting viral infections may be administered directly as a naked nucleic acid construct, preferably further comprising flanking sequences homologous to the host cell genome. When the polynucleotides/vectors are administered as a naked nucleic acid, the amount of nucleic acid administered may typically be in the range of from 1 μg to 10 mg, preferably from 100 μg to 1 mg.

Uptake of naked nucleic acid constructs by mammalian cells is enhanced by several known transfection techniques for example those including the use of transfection agents. Example of these agents include cationic agents (for example calcium phosphate and DEAE-dextran) and lipofectants (for example lipofectam™ and transfectam™). Typically, nucleic acid constructs are mixed with the transfection agent to produce a composition.

Preferably the polynucleotide or vector of the invention is combined with a pharmaceutically acceptable carrier or diluent to produce a pharmaceutical composition. Suitable carriers and diluents include isotonic saline solutions, for example phosphate-buffered saline. The composition may be formulated for parenteral, intramuscular, intravenous, subcutaneous, intraocular or transdermal administration.

The routes of administration and dosages described are intended only as a guide since a skilled practitioner will be able to determine readily the optimum route of administration and dosage for any particular patient and condition.

The invention will be described with reference to the following Examples which are intended to be illustrative only and not limiting. The Examples refer to the above-mentioned Figures.

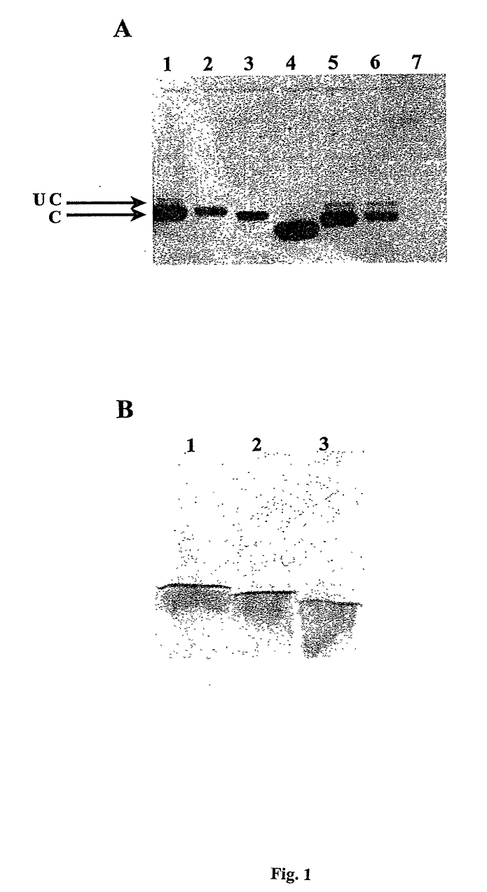

### FIG. 1: Analysis of the Core Proteins Made by the pSFV and pgHCV Constructs

A. Western blot analysis of extracts prepared from cells which were harvested 20 hours after electroporation. Aliquots of extracts containing the same number of cell equivalents were analyzed with antibody JM122. The samples were from cells electroporated with RNA from the following constructs: lane 1, pSFV.1-195; lane 2, pSFV.1-173; lane 3, pSFV.1-169; lane 4, pSFV.1-153; lane 5, pSFV.Δ155-161; lane 6, pSFV.Δ161-166; lane 7, no RNA.

Arrows denote the forms of core which have (labeled C) and have not (labeled UC) been cleaved at the internal processing site.

B. In vitro translation of core proteins. Products of reactions were electrophoresed on a 10% polyacrylamide gel and detected by autoradiography. The samples were from reactions containing the following constructs: lane 1, pgHCV.1-195; lane 2, pgHCV.1-173; lane 3, pgHCV.1-153.

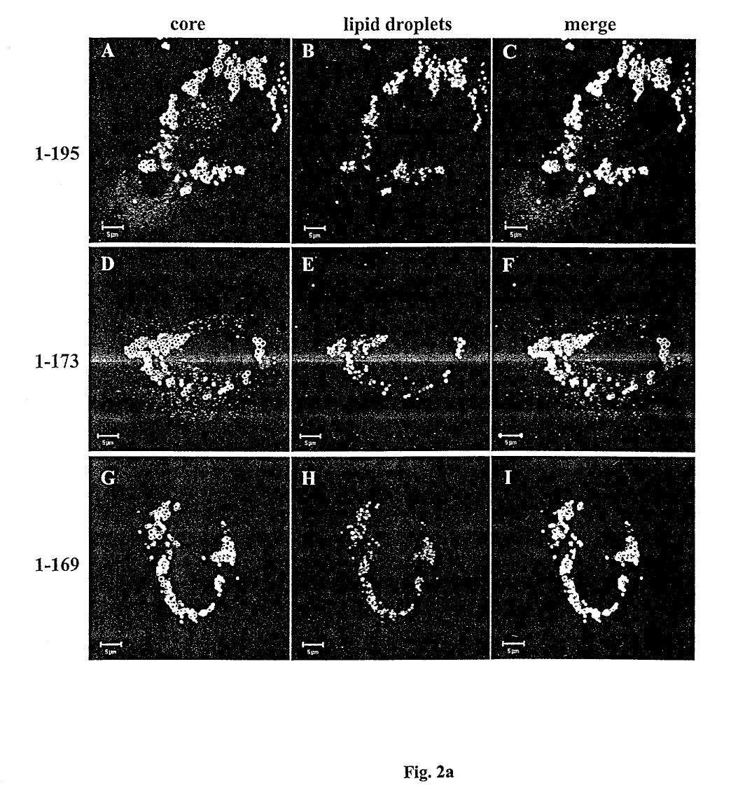

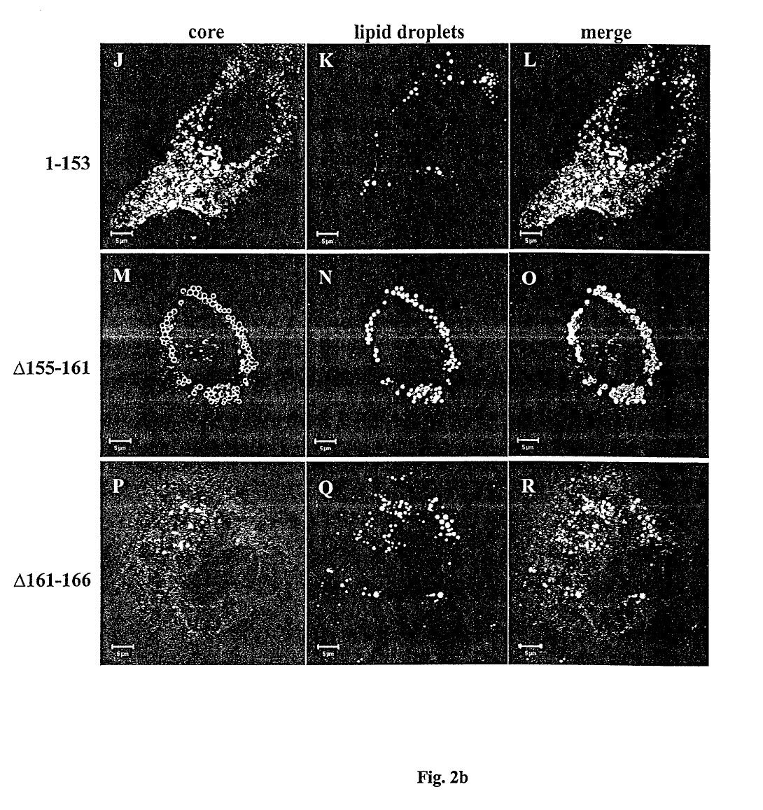

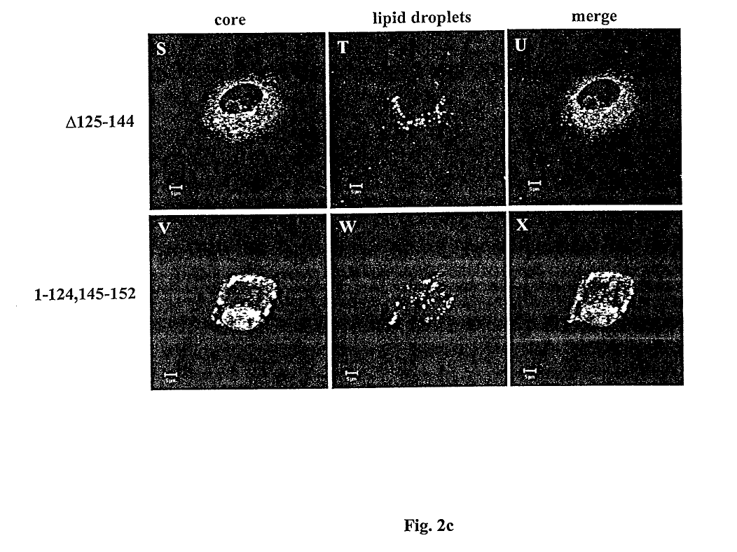

### FIG. 2: Confocal Images of the Intracellular Localization of Core Proteins and Lipid Droplets

BHK C13 cells were harvested 20 hours after electroporation and fixed with 4% paraformaldehyde, 0.1% Triton X-100. Indirect immunofluorescence was performed with antibody JM122 and an anti-mouse secondary antibody conjugated with FITC. Lipid droplets were stained with oil red O. Panels A, D, G, J, M, P, S and V show the distributions of core protein. Panels B, E, H, K, N, Q, T and W show the locations of lipid droplets. Panels C, F, I, L, O, R, U and X are merged images of core protein and lipid droplets. Cells were electroporated with RNA from the following constructs: panels A, B and C, pSFV.1-195; panels D, E and F, pSFV.1-173; panels G, H and I, pSFV.1-169; panels J, K and L, pSFV.1-153; panels M, N and O, pSFV.Δ155-161; panels P, Q and R, pSFV.Δ161-166; panels S, T and U, pSFVA125-144; panels V, W and X, pSFV.1-124, 145-152.

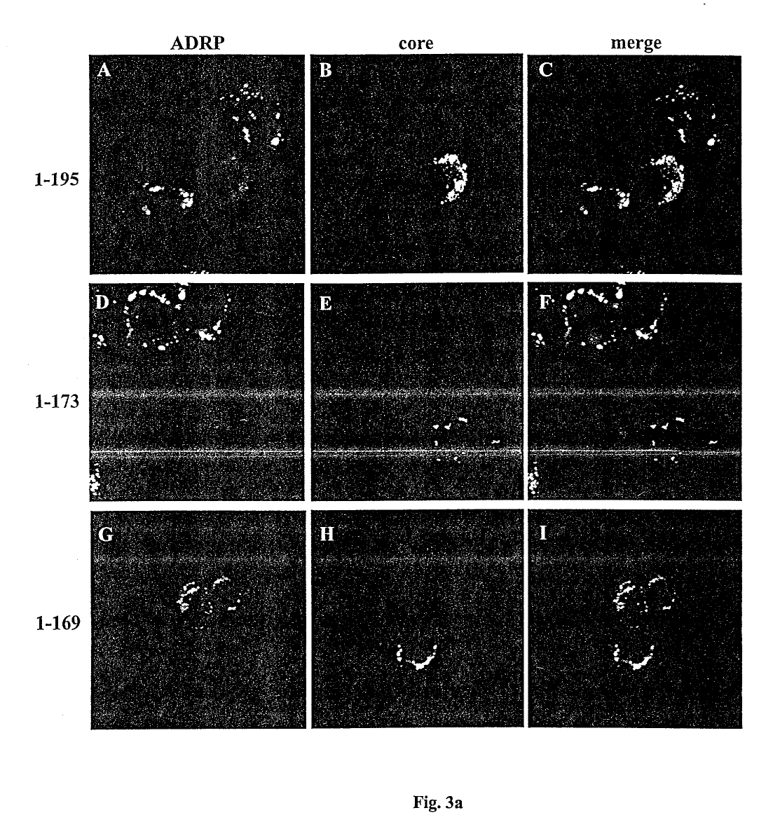

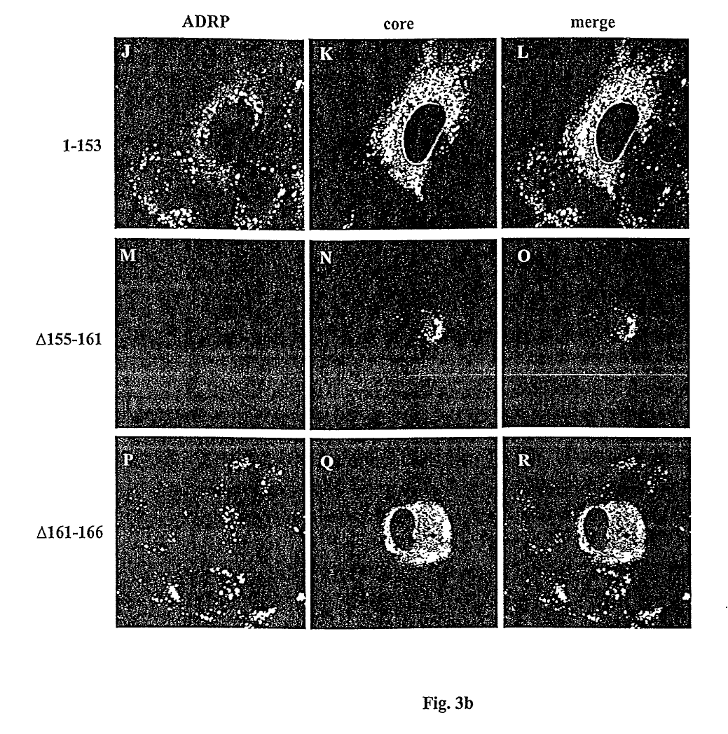

### FIG. 3: Effect of Expression of Core Proteins on the Ability to Detect ADRP in BHKC13 Cells by Confocal Microscopy

Cells were harvested 20 hours after electroporation and fixed with methanol. ADRP was detected with anti-adipophilin antibody and core protein with 308 antisera. Secondary antibodies were an anti-mouse IgG conjugated with FITC (for anti-adipophilin) and anti-rabbit IgG conjugated with Cy5 (for 308 antisera). Panels A, D, G, J, M and P are images of ADRP localization. Panels B, E, H, K, N, and Q are images of core distribution. Panels C, F, I, L, O and R show the merged images of core and ADRP distributions. Cells were electroporated with RNA from the following constructs: panels A, B and C, pSFV.1-195; panels D, E and F, pSFV.1-173; panels G, H and I, pSFV.1-169; panels J, K and L, pSFV.1-153; panels M, N and O, pSFV.Δ155-161; panels P, Q and R, pSFV.Δ161-166.

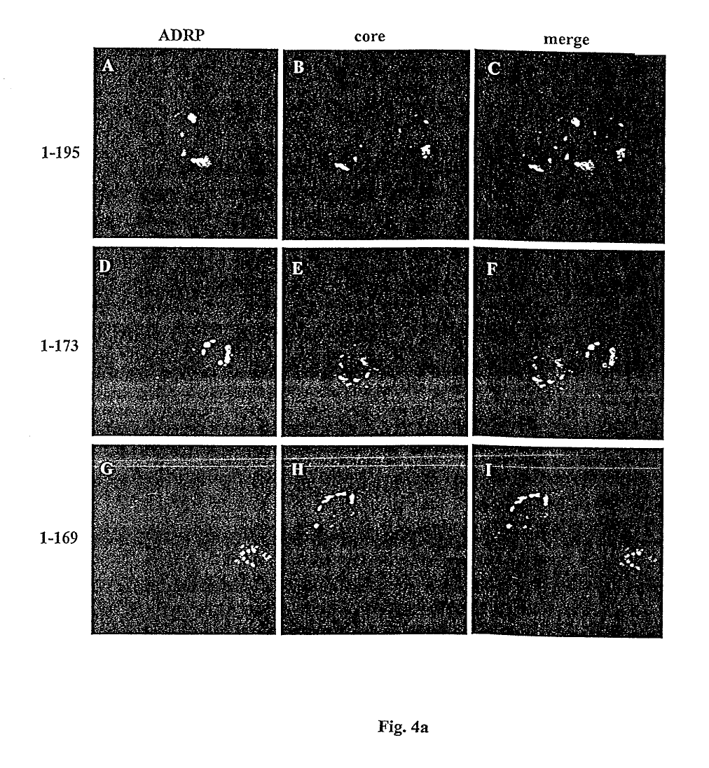

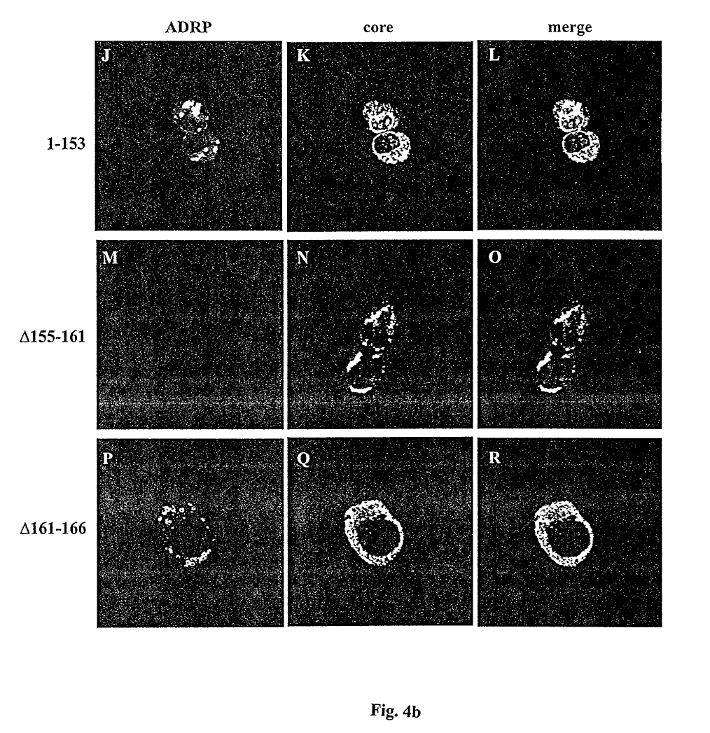

### FIG. 4: Effect of Expression of Core Proteins on the Ability to Detect ADRP in MCA RH7777 Cells by Confocal Microscopy

Cells were examined as described in the legend for FIG. 3.

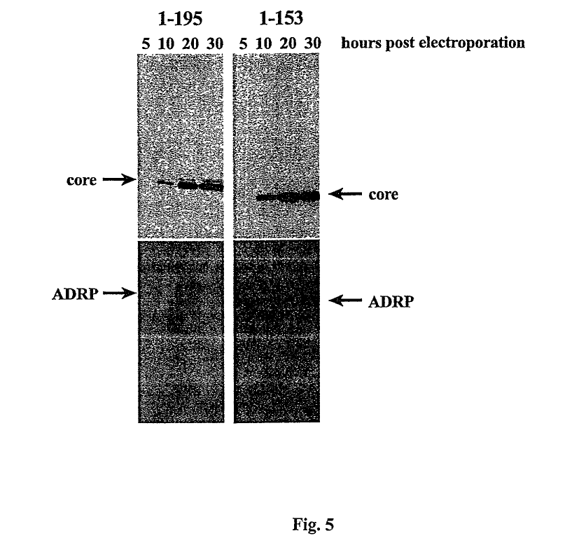

### FIG. 5: Effect of Expression of Core Protein on the Abundance of ADRP

BHK C13 cells were electroporated with RNA from pSFV.1-195 and pSFV.1-153 and extracts were prepared at the times indicated following electroporation. Aliquots of cell extracts were electrophoresed on 10% polyacrylamide gels and then the proteins were transferred to nitrocellulose membrane for Western blot analysis. The upper panels show membranes probed with JM122 antibody while, in the lower panels, membranes were probed with anti-adipophilin antibody. Bands corresponding to core proteins, expressed from pSFV.1-195 and pSFV.1-153, and ADRP are arrowed.

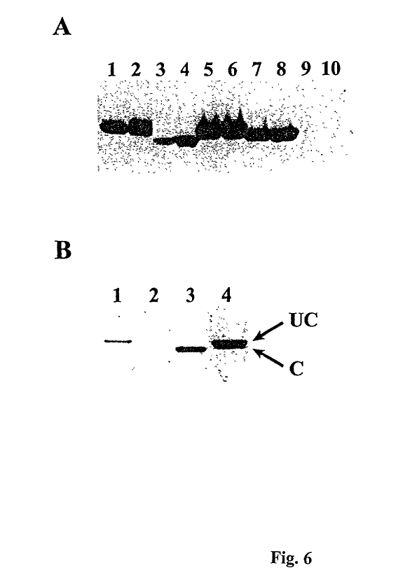

### FIG. 6: Effect of Lipid Globule Targeting Sequences on Stability and Cleavage of Core Protein

A. Western Blot analysis of extracts prepared from cells which were harvested 16 hours after electroporation following treatment with proteasomal inhibitor MG132. Aliquots of extracts containing the same number of cell equivalents were analyzed with antibody JM122. The samples were from cells electroporated with RNA from the following constructs: lanes 1 and 2, PSFV.1-195; lanes 3 and 4, pSFV.1-124, 145-152; lanes 5 and 6, pSFV.Δ125-144; lanes 7 and 8, pSFV.1-153; lanes 9 and 10, no RNA. Samples in lanes with odd numbers were prepared from cells not treated with MG132. Samples in lanes with even numbers were prepared from cells which had been incubated in the presence of MG132 (final concentration 2.5 μM) from 4 hours after electroporation.

B. Western blot analysis of extracts prepared from cells which were harvested 16 hours after electroporation. Aliquots of extracts containing the same number of cell equivalents were analyzed with antibody JM122. The samples were from cells electroporated with RNA from the following constructs: lane 1, pSFV.1-195; lane 2, pSFV.1-124, 145-152; lane 3, pSFV.1-153; lane 4, pSFV.Δ125-144. The two forms of core protein produced by pSFV.Δ125-144 which have been either cleaved or not cleaved (C and UC respectively) at the internal processing site are arrowed.

### EXAMPLES

Cell Lines

Baby hamster kidney (BHK) C13 cells were maintained in Glasgow modified Eagle's medium supplemented with 10% newborn calf serum, 100 IU/ml penicillin/streptomycin and 5% tryptose phosphate broth. The rat hepatoma cell line, MCA RH7777, was maintained in minimal essential Eagle's medium supplemented with 20% foetal bovine serum, 100 IU/ml penicillin/streptomycin, 1 x non-essential amino acids and 2 mM L-glutamine.

Immunological Reagents

Antibody JM122 was a mouse monoclonal antibody raised against a purified fusion protein, expressed in bacteria, which was composed of the N-terminal 118 amino acid residues of core protein encoded by HCV strain Glasgow linked to a histidine tag. Antisera 308 was raised in rabbits against a branched peptide ([A/P]KPQRKTKRNT[I/N]RRPQDVKFPGG)₈K₇A (SEQ ID No. 6). The peptide consists of residues 5-27 of core protein encoded by HCV strain Glasgow (SEQ ID No. 1). The two degenerate sites at positions 1 and 12 were introduced to obtain antisera which would be reactive against core proteins from other isolates. The adipophilin antibody was obtained from Cymbus Biotechnology Ltd.

Secondary antibodies were obtained from Sigma with the exception of Cy5 conjugated goat anti-rabbit IgG which was obtained from Amersham.

Construction of Plasmids

Plasmids containing the coding region for the core protein of HCV strain Glasgow were obtained by combining fragments from two constructs called core.pTZ 18 and 5′-ΔNS2 (provided by M. McElwee and R. Elliott). Core.pTZ18 possesses nucleotide residues 7-615 of SEQ ID No. 1 of the HCV strain Glasgow genome and 5′-ΔNS2 contains residues 1-2895 from reference. DNA fragments from these plasmids were combined in a vector called pGEM1 to give a construct termed pgHCV.CE1E2. This plasmid contains nucleotide residues 337-2895 from reference of the HCV strain Glasgow genome and therefore encodes the core, E1 and E2 proteins of this isolate.

For cloning purposes, the sequences immediately upstream of residue 337 (nucleotide 7 of SEQ ID No. 1) were modified to contain the recognition sequences for Bgl II and Kpn I restriction enzyme sites and immediately downstream of residue 2895, an oligonucleotide was inserted which encodes a translational stop codon followed by the sequences for a Bgl II restriction enzyme site. To create pgHCV.CElE2, a Bgl II DNA fragment containing the core, E1 and E2 sequences was inserted into the Bam HI site of pGEM1; this plasmid was further modified by introducing a Bgl II enzyme site at the Eco RI site in the pGEM backbone. Construction of a derivative plasmid, pgHCV.1-195 was achieved by inserting an oligonucleotide (GCTGAGATCTA—SEQ ID No. 7) that had both a translational stop codon and the sequences for a Bgl II enzyme site between a Fsp I enzyme site at residue 925 (625 of SEQ ID No. 1) in the HCV genome and a Hind III enzyme in the pGEM backbone. Thus, pgHCV.1-195 encodes the N-terminal 195 amino acids of HCV strain Glasgow. The nucleotide and predicted amino acid sequence of this region of HCV strain Glasgow is shown in SEQ ID. No. 1. From pgHCV.1-195, the following series of constructs were made which had various regions of the HCV coding region removed (in 1 to 3 and 6, the numbers following pgHCV represent the amino acid residues of HCV strain Glasgow encoded by each construct):

1. pgHCV.1-173 was constructed by inserting an oligonucleotide GTAACCTTCCTGGTTGCTCTTGAGATCTA (SEQ ID No. 8) between the Bst EII (at nucleotide residue 841 in the HCV strain Glasgow genome) and Hind III enzyme sites (located in the pGEM backbone) in pgHCV.1-195.

2. pgHCV.1-169 was constructed by inserting an oligonucleotide GTAACCTTTGAGATCTA (SEQ ID No. 9) between the Bst EII (at nucleotide residue 841 in the HCV strain Glasgow genome) and Hind III enzyme sites (located in the pGEM backbone) in pgHCV.1-195.

3. pgHCV.1-153 was constructed by inserting an oligonucleotide CTGGCGCATTGAGATCTA (SEQ ID No. 10) between the Bst XI (at nucleotide residue 492 of SEQ ID No. 1) and Hind III enzyme sites (located in the pGEM backbone) in pgHCV.1-195.

4. pgHCV.Δ155-161 was constructed by inserting an oligonucleotide CTGGCCCATGGTGTTAACTATGCAACAG (SEQ ID No. 11) between the Bst XI and Bst EII enzyme sites (at nucleotide residues 492 and 541 of SEQ ID No. 1 respectively in the HCV strain Glasgow genome) in pgHCV.1-195. This construct lacks the nucleotide sequences encoding residues 155 to 161 of the core protein of HCV strain Glasgow.

5. pgHCV.Δ161-166 was constructed by inserting another oligonucleotide CTGGCCCATGGCGTCCGGGTTCTGGAAGACG (SEQ ID No. 12) between the Bst XI and Bst EII sites in pgHCV.1-195. This construct lacks the nucleotide sequences encoding residues 161 to 166 of the core protein of HCV strain Glasgow from reference.

6. pgHCV.1-124, 145-152 was constructed by inserting an oligonucleotide CGATAGAGGCGCTGCCAGGGCCCTGGCGTGAGATCTA (SEQ ID No. 13) between the Cla I (at nucleotide residue 410 from SEQ ID No. 1) and Hind III enzyme sites (located in the pGEM backbone) in pgHCV.1-195.

7. pgHCV.Δ125-144 was constructed by inserting a 400 bp Kpn I/Bst XI DNA fragment from pgHCV.1-124,145-152 (which contains residues 1-124 and 145-152) into a 2970 bp Kpn I/Bst XI DNA fragment from pgHCV.1-195 (which contains residues 153-195).

For expression in tissue culture cells, Bgl II DNA fragments carrying the relevant HCV sequences were prepared from the pgHCV plasmid series and inserted into the Bam HI site of a Semliki Forest virus vector pSFV1. The resultant plasmids were termed the pSFV. series (e.g. pSFV.1-195).

In Vitro Translation

Proteins were translated in vitro using a coupled transcription/translation kit supplied by Promega. Reactions used 1 μg of DNA as template and were carried out according to manufacturer's instructions.

In Vitro Transcription

Prior to electroporation, RNA was transcribed in vitro from the appropriate pSFV plasmid which had been linearized at a Spe I enzyme site. Typical reactions were carried out in a volume of 20 μl and contained 40 mM Tris (pH 7.5), 6 mM MgCl₂, 2 mM spermidine, 10 mM NaCl, 1 mM DTT, 1 mM ATP, 1 mM CTP, 1 mM UTP, 0.5 mM GTP, 1 mM m⁷G(5′)ppp(5′)G cap analogue, 50 units Rnasin, 50 units SP6 RNA polymerase and 2 μg linearized DNA. Reactions were performed at 37° C. for 2 hours. Products of the reaction were analyzed by agarose gel electrophoresis to examine the quality and quantity of RNA synthesized prior to use in electroporations.

Preparation of Cells Competent for Electroporation

Cells were washed and treated with trypsin for detachment from tissue culture containers. Detached cells were suspended in 20 ml of growth medium and centrifuged at 100 g for 5 min at room temperature. Cell pellets were suspended in 50 ml of PBSA and centrifuged as previously. Pellets were suspended in PBSA at a final concentration of about 2×10⁷ cells/ml.

Electroporation of Cells and Preparation of Cell Extracts

0.8 ml of competent cells were mixed with in vitro transcribed RNA in an electroporation cuvette (0.4 cm gap) and pulsed twice at either 1.2 kV, 25 μF (for BHK C13 cells) or 0.36 kV, 960 μF (for MCA RH7777 cells). Between pulses, the cell/RNA suspension was gently mixed. Following electroporation, cells were diluted in growth medium and seeded onto either tissue culture dishes or coverslips in 24-well tissue culture plates and then incubated at 37° C.

To prepare extracts, electroporated cells were harvested by removing the growth medium and washing the cell monolayers with PBS. Cells were scraped into PBS and pelleted by centrifugation at 100 g for 5 mins at 4° C. The cell pellet was solubilized in sample buffer consisting of 160 mM Tris (pH 6.7), 2% SDS, 700 mM β-mercaptoethanol, 10% glycerol, 0.004% bromophenol blue.

Alternatively, sample buffer was added directly to cells which had been washed with PBS. Cells were solubilized at a concentration of approximately 4×10⁶ cell equivalents per ml sample buffer. Samples were heated to 100° C. for 5 mins to fully denature proteins and nucleic acids.

SDS-PAGE and Western Blot Analysis

Samples were prepared for electrophoresis and proteins were separated on polyacrylamide gels cross-linked with 2.5% (wt/wt) N,N′-methylene bisacrylamide using standard techniques. Polypeptides were detected either by autoradiography or by staining using Coomassie brilliant blue.

For Western blot analysis, proteins were separated on polyacrylamide gels and transferred to nitrocellulose membrane using standard techniques. The nitrocellulose membrane was blocked in 3% gelatin, 20 mM Tris (pH 7.5), 500 mM NaCl for at least 6 hours at 37° C. prior to incubation with the primary antibody. Incubations with the primary antibody (diluted to 1/500for adipophilin antibody and 1/1000for JM122) were performed in 1% gelatin, 20 mM Tris (pH 7.5), 500 mM NaCl, 0.05% Tween 20 at either room temperature or 37° C. for approximately 3-4 hours. Following extensive washing with 20 mM Tris (pH 7.5), 500 mM NaCl, 0.05% Tween 20, the membrane was incubated for 2 hours at room temperature with anti-mouse IgG conjugated with horse radish peroxidase in the same solution as for the primary antibody and at a dilution of 1/1000. Bound antibody was detected by enhanced chemiluminescence.

Indirect Immunofluorescence and Staining of Lipids

Cells on 13 mm coverslips were fixed in either methanol at −20° C. or 4% paraformaldehyde, 0.1% Triton X-100 (prepared in PBS) at 4° C. for 30 mins. Following washing with PBS and blocking with PBS/CS (PBS containing 1% newborn calf serum), cells were incubated with primary antibody (diluted in PBS/CS at 1/200 for JM122 antibody, 1/1000 for 308 antisera and 1/100 for adipophilin antibody) for 2 hours at room temperature. Cells were washed extensively with PBS/CS and then incubated with conjugated secondary antibody (either anti-mouse or anti-rabbit IgG raised in goat) for 2 hours at room temperature. Cells were washed extensively in solutions of PBS/CS followed by PBS and finally H₂O before mounting on slides using Citifluor. Samples were analyzed using a Leiss LSM confocal microscope.

Following incubation with both antibodies and washing, lipid droplets were stained in paraformaldehyde-fixed cells by briefly rinsing coverslips in 60% propan-2-ol followed by incubation with 0.5 ml 60% propan-2-ol containing oil red O for 1.5-2 mins at room temperature. Coverslips were briefly rinsed with 60% propan-2-ol, washed with PBS and H₂O and mounted as described above. The oil red O staining solution was prepared from a saturated stock of approximately 1% oil red O dissolved in propan-2-ol. Before staining, the stock was diluted with H₂O and then filtered.

### RESULTS

### Example 1

### Expression of HCV Core Protein and Variants in Tissue Culture Cells

Presently, there is no system available for propagating HCV in tissue culture cells. Therefore, expression of HCV gene products necessitates the use of heterologous expression systems. For short-term expression in mammalian cells, a variety of viral vectors have been utilized including vaccinia virus, Sendai virus and adenovirus. A further alternative is the Semliki Forest virus (SFV) system in which in vitro transcribed RNA, that encodes the SFV replication proteins as well as a heterologous protein but not the SFV structural proteins, is introduced into tissue culture cells. Introduction of nucleic acid into cells may be achieved by several routes but, in the examples given, the method of choice is electroporation.

BHK cells were electroporated with RNA from the series of pSFV constructs. 20 hours following electroporation, cells were harvested and extracts prepared. Samples were electrophoresed on a 10% polyacrylamide gel and, following electrophoresis, the proteins were transferred to nitrocellulose membrane for Western blot analysis.

Probing the membrane with the core-specific monoclonal antibody JM122 revealed a major single species in each sample which corresponds to core protein. The apparent molecular weights of the proteins made by pSFV.1-195 and two truncated variants, pSFV.1-173 and pSFV.1-169, are approximately 21 kDa and are almost identical (FIG. 1A, lanes 1-3). Cleavage between the core and E1 coding regions occurs between residues 191 and 192. However, there is additional data which reveals that the core protein is further processed by cleavage around residue 174 (Moradpour et al., 1996) this cleavage site will be referred to as the internal processing site. The precise residue at which this second cleavage event occurs is not known. Hence, in agreement with FIG. 1A, lanes 1-3, it would be predicted that the three constructs named above would generate products of similar molecular weights.

Additional evidence for a cleavage event close to residues 169-173 occurring within tissue culture cells is shown in FIG. 1B. Here, polypeptides translated in vitro from the pGEM versions of 3 core variants reveal that the unprocessed species made from pgHCV.1-195 is larger than that from pgHCV.1-173 (compare lanes 1 and 2). As would be predicted from the coding sequences for the third truncated form of core, the major species synthesized from pSFV.1-153 has a lower apparent molecular weight than that from pSFV.1-195 (FIG. 1A, compare lanes 1 and 4). The major species made by the internal deletion mutants pSFV.Δ155-161 and pSFV.Δ161-166 are intermediate in size between those produced by pSFV.1-195 and pSFV.1-153 (FIG. 1A, lanes 5 and 6). Again, this agrees well with predictions based on the number of amino acids removed in these variants (7 in pSFV.Δ155-161 and 6 in pSFV.Δ161-166). It is also evident that there is a significant amount of material produced by the two internal deletion variants which has a higher molecular weight than the fully processed form of core. This presumably represents reduced cleavage at the internal processing site which may result from the removal of certain residues in these mutants which are necessary for fully efficient processing. To conclude, the core proteins and its variants produced by the SFV constructs can be detected by a core-specific antibody and their apparent molecular weights are in agreement with predictions from the nucleotide sequences and previously published data.

### Example 2

### Intracellular Distribution of HCV Core Protein

Previous studies have revealed that the HCV core protein can associate with lipid droplets within the cytoplasm of cells (Barba, G. et al., 1997; Moradpour, D. et al., 1996). This conclusion was arrived at by combining the techniques of immune electron microscopy with the ability to stain lipid with osmium tetroxide. However, this method suffers from the disadvantages that it is time-consuming and osmium tetroxide can stain other biological molecules (e.g. proteins) in addition to lipid. Therefore, we developed a method for detecting proteins firstly by indirect immunofluorescence followed by staining of lipid droplets with the oil-soluble colorant oil red O. Combined with the method of confocal microscopy, it is possible to visualize the intracellular localizations of core protein and lipid droplets separately and together. A typical example is shown in FIG. 2, panels A-C. Here, BHK C13 cells have been electroporated with pSFV.1-195 RNA and, following incubation at 37° C. for 20 hours, the cells have been examined by both indirect immunofluorescence and staining with oil red O. In panel A, the core protein produced by pSFV.1-195 is seen to locate to vesicular structures in the cytoplasm. Panel B reveals the distribution of lipid droplets in the same cell. By merging these data (panel C), it is evident that core protein is sited around the lipid droplets. These data therefore agree with previously published results for constructs expressing the full-length coding region of core.

### Example 3

### Association of HCV Core Protein with Intracellular Lipid Droplets Requires Amino Acids 161 to 166 and 125 to 144

Results with the constructs which produce truncated forms of core protein indicate that proteins consisting of 173 and 169 amino acids of the core coding region also locate to droplets (FIG. 2, panels D-I). By contrast, expressing only the N-terminal 153 residues results in loss of localization to droplets and a diffuse cytoplasmic distribution is observed (FIG. 2, panels J-L). Thus, residues of core protein between amino acids 154 and 169 are required for localization to droplets. Studies with the internal deletion mutants pSFV.Δ155-161 and pSFV.Δ161-166 further examined segments within this 16 amino acid region which may be important for core protein localization. From the resultant data, removal of residues between 155 and 161 did not affect lipid droplet association whereas removal of residues between 161 and 166 gave a diffuse cytoplasmic pattern (FIG. 2, compare panels M-O with P-R). Hence, between residues 154 and 169, amino acids from 161 to 166 play an essential role in the ability of core protein to locate to lipid droplets.

Further analysis of other internal deletion mutants (which removed residues 9-43, 4-75 and 80-118) showed that the core proteins made by these constructs continued to associate with lipid droplets (data not shown). Hence, these regions are dispensable for binding to droplets. However, a construct expressing a core variant in which residues 125 to 144 had been deleted failed to distribute to droplets and gave a diffuse cytoplasmic fluorescence (FIG. 2, panels S, T and U). This mutant therefore identifies a second region in addition to the segment between 161 and 166 which is necessary for association with droplets. The data suggest that both sets of sequences are required for targeting to lipid droplets. In agreement with these data, a core variant which is truncated at residue 152 and lacks amino acids 125 to 144 also fails to bind to droplets (FIG. 2, panels V, W and X). Additionally, this protein, in which both sets of targeting sequences are deleted, is present in low amounts in electroporated cells as a consequence of degradation.

### Example 4

### Effect of Localization of Core Protein on the Lipid Droplet Associated Protein ADRP

At present, there are few proteins identified in mammalian cells which are known to associate with lipid droplets. One protein which has been recently identified is ADRP which is ubiquitously expressed in a number of tissue culture cell lines; ADRP MRNA has also been detected in a range of tissue types in mice. To examine whether the localization of core to lipid droplets had any affect on ADRP, BHK C13 cells were electroporated with the series of pSFV constructs expressing core protein and its variants. An example of the data is shown in FIG. 3. Panels A to C show images of three cells following electroporation with pSFV.1-195, only one of which contains core protein (Panel B). Immunofluorescent results with the adipophilin antibody (panel A) reveal that ADRP is located on vesicular structures, consistent with its previously assigned association with lipid droplets. The protein is readily detected in the cells which do not express core protein, however, it is considerably reduced in abundance in the core-expressing cell. Observations from this and a series of other experiments consistently revealed that cells expressing core protein from pSFV.1-195 either lacked or contained barely detectable amounts of ADRP. Nonetheless, some cells in which both core protein and ADRP were present also were found; in general, such cells gave reduced fluorescence for the core protein. Hence, it was concluded that the loss of ADRP was related to the levels of expression of core in individual cells. Results with the variants of core which continue to locate to lipid droplets gave identical data (see panels D-I and M-O). Thus, the majority of cells expressing core protein from constructs pSFV.1-173, pSFV.1-169 and pSFV.Δ155-161 contained quantities of ADRP which were barely detectable. By contrast, ADRP continued to be readily found in cells producing core proteins from pSFV.1-153 and pSFV.Δ161-166, the variants which do not associate with lipid droplets (see panels J-L and P-R). Thus, association of core protein with lipid droplets correlates with a loss of ability to detect ADRP by immunofluorescence. Any cell type specificity for this affect was tested by performing identical experiments in the rat hepatoma cell line, MCA RH7777. In these cells, core protein and its variants gave identical results for their ability to associate with lipid droplets and this again correlated with the levels of ADRP detected in core-expressing cells (FIG. 4). Thus the effect of core protein on ADRP is not cell-type specific.

### Example 5

### The Ability of Core Protein to Associate with Lipid Droplets Induces a Loss of ADRP

The immunofluorescence data revealed that the association of core protein and its variants with lipid droplets led to an inability to detect ADRP. It was possible that this was due to masking of ADRP by core. To examine directly the effect of core protein on the levels of ADRP, Western blot analysis was performed on cell extracts prepared at various times following electroporation with either pSFV.1-195 or pSFV.1-153 RNA. In parallel, immunofluorescence analysis was also performed on these cells and this revealed that expression of the core protein produced by the two RNAs was apparent in greater than 90% of cells. Analysis with antibody JM122 indicated that core protein could be detected from both constructs at 10 hours following electroporation and peaked by about 20 hours (FIG. 5). The abundance of core protein produced by the two constructs was very similar by this time-point. From analysis of these samples with the ADRP-specific antibody, it is apparent that there is no change in the abundance of ADRP following electroporation with the pSFV. 1-153 RNA. A third set of cells in this experiment which was electroporated with SFV RNA which expresses the HCV E1 and E2 proteins also showed no reduction in ADRP levels with time. By contrast, there is a rapid reduction in ADRP levels to barely detectable quantities which mirrors the rise in core protein made from pSFV.1-195.

From staining of polyacrylamide gels with Coomassie brilliant blue, there were approximately equivalent amounts of protein in all samples. In addition, probing the membranes with another antibody for a endoplasmic reticulum-specific protein, calnexin, indicated that both pSFV.1-195 and pSFV.1-153 samples had similar quantities of this protein at the various times following electroporation. This affect of core expression on ADRP was consistently found in other experiments. Thus, the association of core protein with lipid droplets directly correlates with a specific reduction in the abundance of this protein in cells.

### Example 6

### Removal of Lipid Globule Targeting Sequences Reduces Protein Stability and Impairs Cleavage at the Internal Processing Site

Immunofluorescence data with pSFV.1-124,145-152 and pSFV.Δ125-144 showed that the proteins made by these constructs gave diffuse fluorescence. In two separate experiments, Western blot analysis of extracts prepared from cells electroporated with RNA from these constructs revealed significantly reduced amounts of protein made by pSFV.1-124,145-152 as compared to that made by pSFV.1-195, pSFV.1-153 and pSFV.Δ125-144 (FIG. 6A, compare lane 3 with lanes 1, 5 and 7 and FIG. 6B, compare lane 2 with lanes 1, 3 and 4).

The level of reduction was approximately 5-fold. This low level of detectable protein was not the result of reduced amounts of RNA synthesized by in vitro reactions (data not shown). Addition of the proteasome inhibitor MG132 to a parallel culture of cells electroporated with pSFV.1-124, 145-152 gave rise to a significant (4-fold) increase in the amount of protein detected by Western blot analysis (FIG. 6A, compare lane 4 with lane 3). Therefore, the reduction in protein levels from pSFV.1-124, 145-152 RNA is apparently not due to inefficient translation. These data suggest that removal of both sets of sequences which are required for targeting to lipid droplets induces rapid degradation of the protein, analogous to that observed for ADRP in the presence of full-length core protein.

It was observed also that two protein products derived from pSFV.Δ125-144 were detected by antibody JM122. From their mobilities, it was assumed that the upper band represented protein which had not been cleaved at the internal processing site (labeled UC in FIG. 6B, lane 4) while the lower band was processed (labeled C in FIG. 6B). Examination of the relative intensities of these species indicates that the uncleaved product is more abundant (approximately 4-5 fold) than the cleaved material. This provides evidence that the sequences between 125 and 144, which are required for lipid droplet association also influence the efficiency of cleavage at the internal processing site. Cleavage at this site is mediated by a cellular signalase which may be membrane bound. Hence, disruption of the targeting function of these residues is likely to inhibit cleavage at the internal processing site and, from evidence of the maturation of related pestiviruses and flaviviruses, such an effect will reduce virus maturation and growth.

### Example 7

### In Vitro Binding Assay to Test Effects of Candidate Substances Synthesis of Lipid Targeting Sequences and ADRP Using in Vitro Transcription/Translation

Constructs suitable for in vitro synthesis of mRNA are produced by placing nucleotide sequences encoding core protein (e.g. pgHCV.1-195) or any other protein with core lipid globule targeting sequences (e.g. pgHCV.Δ43-119) and ADRP are placed under the control of a suitable bacterial or bacteriophage RNA polymerase promoter e.g. SP6 or T7. RNA transcripts are prepared from each of these constructs using standard methods and the yields of RNA determined.

To produce in vitro translated protein, the in vitro synthesized RNA is added to an extract capable of synthesizing polypeptides e.g. a reticulocyte lysate prepared from rabbits or an extract from wheat germ; proteins may be radiolabeled by including an isotopically labeled amino acid such as ³⁵S-methionine.

Assessing Binding of Proteins to Lipid

Membranous material prepared from animal cells (e.g. microsomal membranes) or synthetically prepared mixtures of triacylglycerol or cholesterol and phospholipid are mixed with the translated proteins and incubated. Lipid fractions are then recovered by centrifugation. The incorporation of proteins into lipid fractions may be determined either directly by SDS-PAGE of radiolabeled proteins, or indirectly by using antibodies which are functional in Western blot and/or ELISA procedures.

Combinations of RNA from core protein and ADRP may be mixed in the same reaction in various proportions to test the relative affinity of each protein for lipid. An example of a combination would be 9 parts pgHCV.1-195 RNA to 1 part ADRP RNA.

Candidate substances at a range of concentrations, such as from 1 μM to 100 mM, are added to reactions and the effect of these substances on protein binding to lipid analyzed using the methods indicated above. A suitable candidate substance is typically a substance that enhances or does not significantly impair lipid association of ADRP but which reduces or abolishes binding of core to lipid.

### Example 8

### In Vivo Binding Assay to Test Effects of Candidate Substances

BHK C13 cells are electroporated with SFV encoding HCV core protein (for example SFV.1-195) as described above. At various time points after electroporation, candidate substances are added to cells at concentrations which are not cytotoxic (as determined with mock electroporated cells, for example). Following incubation at 37° C., cells are harvested and cell extracts prepared. Extracts are analyzed by Western blot analysis (antibody JM122) to examine the abundance of core protein and determine the efficiency of cleavage at the internal processing site. The relative abundance of core protein in treated as compared to untreated cells could also be quantitated by ELISA.

In addition, cells are fixed and examined by a combination of indirect immunofluorescence (for the core and ADRP proteins) and oil red O staining (for lipid droplets) as described above. This can be used to determine whether the intracellular distribution and abundance of core and ADRP has been altered by the presence of the candidate substances. Western blot analysis may also be used to confirm whether the candidate substance had any affect on ADRP levels.

A suitable candidate substance is typically a substance that prevents or reduces core association with droplets, causes reduced levels of core protein and/or impaired cleavage at the internal processing site. By contrast, the candidate substance should preferably not affect the intracellular distribution of ADRP and its abundance is typically either similar to or higher than in cells that do not express the core protein.

### Example 9

### Assessing Anti-viral Affects of Candidate Substances in Transgenic Animals Expressing Core or in Chimpanzees

In transgenic animals expressing liver-specific core protein or animals (chimpanzees) infected with HCV, test substances are added at concentrations which were not toxic to the host.

Transgenic Animals Expressing Core Protein in a Liver-specific Manner

Transgenic mice which give liver-specific expression of core protein are known in the art. Expression of core protein is associated with the development of steatosis and hepatocellular carcinoma in two lines of such animals (Moriya, K. et al., 1998 and Moriya, K. et al., 1997); both pathologies are associated with HCV infection. Similar transgenic mice may be produced with similar phenotypes and used to examine the effect of substances which prevent core association with lipid droplets on these pathological changes.

The effect of candidate substances on core protein localization and levels may be determined in transgenic animals using cells obtained by liver biopsies and tested using the techniques described in Example 8. Alternatively, or in addition, the effect of a candidate substance of the development of steatosis and hepatocellular carcinoma in these animals may be determined. The efficacy of candidate substances may be measured by a reduction in the pathological changes which occur e.g. reduced hepatosteatosis and significant delays or prevention of the onset of carcinoma.

Chimpanzees Infected with HCV

The effect of a substance on HCV replication in infected chimpanzees is assessed by RT-PCR analysis of sera taken at regular intervals from the animal. Biopsy material from the liver may also be tested for the presence of negative-strand HCV RNA by RT-PCR using standard techniques (see Conry-Cantilena, 1997 for review and references contained therein). Efficacy of the candidate substance is assessed by the reduction in the levels of HCV RNA as measured in either or both assays. A reduction in viral titer by a factor of at least 2 to 3 logs is indicative of an anti-viral effect.

### Example 10

### Identification of a Domain Required to Direct Core Protein of HCV and GB Virus B to Lipid Droplets

In this example we have identified a conserved domain that is present in equivalent structural proteins encoded by HCV and GBV-B and directs these proteins to lipid storage compartments.

GBV-B is a virus called GB virus-B. Information on GB virus-B has been presented by Beames et al (2000, Journal of Virology vol 74 No. 24, pp 11764-11772) and Lanford et al (2001, Journal of Virology vol 75 No. 17, pp 8074-8081).

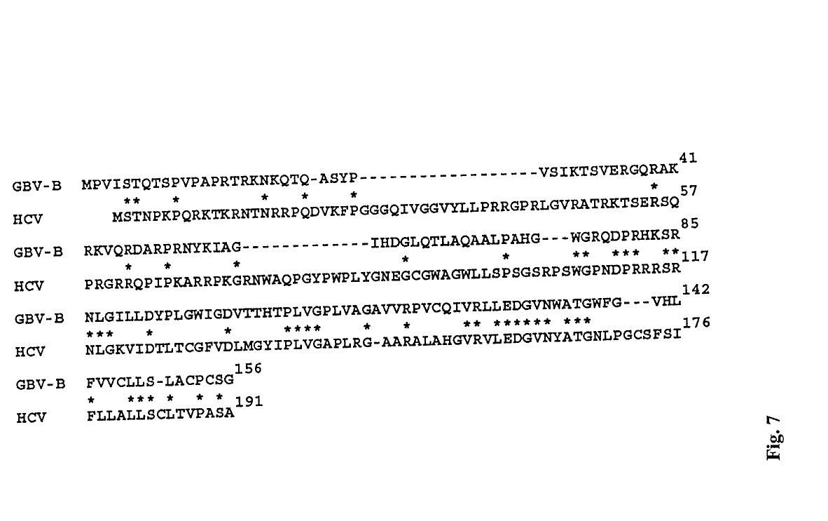

GBV-B was isolated from tamarins but the natural host of the virus is not known. The GBV-B core protein is described in the example below. The nucleic acid sequence of GBV-B is shown in Genbank Accession No. NC001655. GBV-B is the closest known related virus to HCV in terms of sequence identity (29). GBV-B core protein is an example of a homologue of HCV core protein. An alignment of HCV and GVB-B amino acid sequence is shown in FIG. 7. Furthermore, GVB-B is an example of a lipid globule targeting sequence.

Lipid droplets are intracellular storage organelles that are found in all eukaryotic organisms and some prokaryotes (reviewed in 1-3). They consist of a core of neutral lipid, comprising mainly triacylglycerols and/or cholesterol esters, surrounded by a monolayer of phospholipids. Bounding the phospholipid layer is a proteinaceous coat. In mammalian cells, the principal lipid droplet binding proteins that have been identified are adipophilin (also called adipocyte differentiation-related protein, ADRP; 7, 8) and a related family of proteins termed the perilipins (9-11). Adipophilin is present in a wide range of cell types and in increased quantities in certain diseases where intracellular lipid accumulation is evident (12-14). By contrast, expression of the perilipins is restricted to adipocytes and steroidogenic cells (15, 16).

In addition to adipophilin and the perilipins, the core protein encoded by hepatitis C virus (HCV) also associates with lipid droplets in mammalian cells (17-19). HCV is the sole member of the hepacivirus genus that is incorporated into the Flaviviridae family along with two other genera, the flavi- and pestiviruses. All of the Flaviviridae are positive-sense, single stranded RNA viruses that have similar genome arrangements and share sequence similarities. HCV core is a structural component of the virus particle and, by analogy with flavi- and pestiviruses, it is likely to be the sole component of the capsid (20, 21). The protein is generated from a polyprotein encoded by the viral genome by cleavage at the endoplasmic reticulum (ER; 22-26). Expression of core can lead to the genesis of lipid droplets in tissue culture cells (18) and the development of steatosis in transgenic mice (27). Moreover, interaction between core and apolipoprotein AII, a component of lipid droplets, has been demonstrated (28). These data indicate that, not only does core associate with lipid droplets, but it also may have the capacity to influence metabolic events within the cell involving the storage of lipid.

To understand the significance of the interaction between HCV core and lipid droplets, a region within the viral protein that is essential for its association with these storage organelles had been identified (19). In this example, studies have been carried out to determine whether there is any similarity between the sequences within this region and those of GBV-B, which has significant sequence identity to HCV although, as yet, it remains unclassified among the genera of the Flaviviridae (29). GBV-B shares a tropism for liver hepatocytes with HCV and is infectious in tamarins (30, 31). Thus, it has been suggested that GBV-B infection of tamarins may be a surrogate model system for HCV infection of humans (31). However, few comparative studies between equivalent proteins encoded by the two viruses have been conducted.

Construction of Plasmids

Construction of plasmids pSFV/1-195 and pSFV/1-169 has been described previously (19). The codons for the proline residues in these constructs were mutated to encode alanine by insertion of an oligonucleotide (HCV1 in Table 1). This oligonucleotide was inserted between BspHI and BstXI sites in construct pgHCV/135-144 (19) to give plasmid pgHCV/1-195_{(P A)} [symbol]; the BspHI site is not a natural site in the sequence for HCV strain Glasgow and was introduced during the construction of pgHCV/135-144. To generate pSFV/1-195_{(P A)} [symbol] and pSFV/1-169_{(P A)} [symbol], a 502bp fragment from pgHCV/1-195_{(P A)} [symbol], produced by cleavage by BglII and BstEII, was inserted into pSFV/1-195 and pSFV/1-169 digested with the same enzymes. A tagged version of HCV core was generated by firstly converting the nucleotide sequence in pg/1-195 that encodes amino acids 116 and 117 from TCG CGC to TCT AGA; this introduced a novel XbaI site into the HCV core-coding region without changing the encoded amino acids. An oligonucleotide (HCV2 in Table 1) that encoded the epitope tag (32) was introduced into the XbaI site to give plasmid pg/1-195tag. The tagged version of core was transferred as a BglII fragment from pg/1-195tag into Semliki Forest virus (SFV) expression vector pSFV1 (33) to give construct pSFV/1-195tag.

TABLE 1
Oligonucleotides used to generate constructs.

| | | Nucleotide |
|---|---|---|
| Name | Oligonucleotide Sequence | Position^{a} |
| HCV | CATGGGGTACATAGCGCTCGTCGGCGCCGCCT | |
| 1 | TAAGAGGCGCTGCGAGGGCC (SEQ ID No. 14) | |
| HCV | CTAGAGAGCGCAAGACGCCCCGCGTCACCGG | |
| 2 | CGGCG (SEQ ID No. 15) | |
| GBV | GGAGATCTCGTAGACCGTAGCACATG | 428-448 |
| B1 | (SEQ ID No. 16) | |
| GBV | GGGGATCCCTAGTGGACACCGAACCAACCAG | 842-868 |
| B2 | TAGCCCA (SEQ ID No. 17) | |
| GBV | GGGGATCCTCAGATCACACAACCAGGCTCGT | 1003-1029 |
| B3 | GTAGG (SEQ ID No. 18) | |
| GBV | GGGTACTCTAGAGTGATAGGCCTGGTC | 1618-1639 |
| B4 | (SEQ ID No. 19) | |
| GBV | CTAGAGAGCGCAAGACGCCGCGGGTCACCGG | |
| B5 | TGGCTCTCGCAATCTTGG (SEQ ID No. 20) | |

^{a}Nucleotide positions on the GBV-B viral genome (Genbank Accession No. NC001655) for the GBV-B primers

To express portions of the GBV-B polyprotein, relevant regions were amplified by PCR from a construct pGBB (30). pGBB contains the consensus sequence for an infectious molecular clone of GBV-B (30). Primers for PCR amplification were derived from the viral sequences in pGBB and were used in the following pairs to produce DNA fragments that encoded N-terminal regions of the GBV-B polyprotein: residues 1-141, GBV-B primers 1 and 2; residues 1-194, GBV-B primers 1 and 3; residues 1-398, GBV-B primers 1 and 4. Amplified fragments were introduced initially into plasmid pGEM1 and thereafter into pSFV1 by standard cloning techniques. To permit detection of GBV-B core, an epitope tag (32) was introduced into the coding region immediately following amino acid residue 85 (FIG. 7). This was accomplished by firstly introducing a novel XbaI site at nucleotide residue 695 by converting the sequence from TCT CGC to TCT AGA; this did not alter the encoded amino acid sequence. An oligonucleotide encoding the epitope tag (GBV-B5 in Table 1) was inserted between this XbaI site and a TfiI site (position 708 in the native GBV-B nucleotide sequence). The final SFV constructs that contained tagged versions of GBV-B core were termed pSFV/GB1-141, pSFV/GB1-194 and pSFV/GB1-398.

Maintenance of Tissue Culture Cells and Treatment with MG132

Baby hamster kidney (BHK) C13 cells were grown and maintained in Glasgow minimal Eagle's Medium supplemented with 10% new-born calf serum (CS), 4% tryptose phosphate broth, and 100 IU/ml penicillin/streptomycin (ETC10). To treat BHK cells with MG132 (supplied by Boston Biochem), cells were incubated for 5h after electroporation at 37° C. and the media was replaced with fresh media containing the protease inhibitor at a final concentration of 2.5 μg/ml. Incubation was continued at 37° C. for a further 12 h before the cells were either harvested for Western blot analysis or fixed for indirect immunofluorescence studies.

Immunological Reagents

The monoclonal antibodies (MAb) used to detect HCV core protein (MAb JM122) and the epitope tag (MAb 9220) have been described previously (19 and 32 respectively).

In Vitro Transcription and Electroporation of SFV RNA into Cells

RNA was transcribed in vitro from recombinant pSFV constructs linearized with SpeI. BHK cells were electroporated with in vitro transcribed RNA as described previously (19, 36). Cells were incubated at 37° C. for 15h and then harvested.

Preparation of Cell Extracts, Polyacrylamide Gel Electrophoresis and Western Blot Analysis

Extracts were prepared and polyacrylamide gel electrophoresis performed as described previously (19, 36).

For Western blot analysis, proteins separated on polyacrylamide gels were transferred to nitrocellulose membrane. After blocking with 3% gelatin, 4 mM Tris-HCl, pH 7.4, 100 mM NaCl, membranes were incubated with antibodies (diluted to 1/500) in 1% gelatin, 4 mM Tris-HCl, pH 7.4, 100 mM NaCl, 0.05% Tween 20. After washing, bound antibody was detected using a horseradish peroxidase-conjugated secondary antibody followed by enhanced chemiluminescence (Amersham, UK).

Indirect Immunofluorescence and Staining of Lipids

Cells on 13 mm coverslips were fixed for 30 min in 4% paraformaldehyde, 0.1% Triton X-100 (prepared in phosphate-buffered saline) at 4° C. Following washing with phosphate-buffered saline (PBS) and blocking with PBS/CS (PBS containing 1% newborn calf serum), cells were incubated with primary antibody (diluted in PBS/CS at 1/200for JM122 and 9220 antibodies) for 2h at room temperature. Cells were washed extensively with PBS/CS and then incubated with conjugated secondary antibody (either anti-mouse or anti-rabbit IgG raised in goat) for 2 h at room temperature. Cells were washed extensively in solutions of PBS/CS followed by PBS. Staining of lipid droplets by oil red O was performed as described previously (19). Cells were rinsed finally with H₂O before mounting on slides using Citifluor (Citifluor Ltd., UK). Samples were analyzed using a Zeiss LSM confocal microscope.

Computing

Sequences were aligned using the CLUSTAL W alignment program (37) and hydropathicity plots generated by ProtScale (38).

### RESULTS

A Domain in the HCV Core Protein that is Absent in Flavi- and Pestiviruses but Present in GBV-B

Previously, it was proposed that the HCV core protein consisted of 3 domains and that the second of these domains was absent in related pesti- and flaviviruses (19, 39). Although there is a lack of sequence identity between viral sequences in the Flaviviridae, each of the capsid proteins in members of this virus family have a high content of basic amino acids (40). Therefore, the comparisons described here were based on the proportion of positively charged residues in predicted coding regions accompanied by hydropathicity plot studies. It was found that the mature capsid (C) protein of yellow fever virus (YF), a flavivirus, had a high proportion of basic residues (27%). In contrast, the signal peptide sequence that is removed from C protein upon maturation did not contain any basic amino acids. Up to amino acid 117 of the HCV core protein, 23% of residues were basic and this dropped to 7% between residues 118-173. Thus, the N-terminal 117 amino acids of HCV core have a similar character to those in the YF C protein but there are no sequences corresponding to the region between 118 and 173 of the HCV polypeptide. Analysis of the hydropathicity of the HCV core protein also revealed a highly hydrophilic region containing a similar segment of basic residues (RRRSR) between residues 113-117 (FIG. 7). The region between residues 118 and 173 is referred to as domain 2 (19, 39).

A closely related virus to HCV is GBV-B, a virus that was isolated from tamarins but whose natural host is not known. To date, the proteolytic events to generate the mature proteins of GBV-B have been assumed from comparison with HCV polypeptide processing (29). Comparing the putative GBV-B core sequence with that of HCV did not identify any stretches of similarity until residue 75 of GBV-B (FIG. 7). According to the sequence alignment, domain 1 for GBV-B core ended at residue 85 (corresponding to residue 117 in HCV core; FIG. 7) and thus, for this domain, the GBV-B sequence was shorter than that for HCV. The overall sequence identity (sequence homology) between these domains in HCV and GBV-B was about 22%. From residue 86 and up to residue 140 of GBV-B, sequence identity between the two viral sequences increased to 41% and apart from one additional residue in GBV-B, the sequences were colinear (FIG. 7). In HCV core, this segment is composed principally of domain 2 and indicated that sequences corresponding to this region are present in GBV-B. Sequence identity between the residues 140-156 for GBV-B and 174-191 for HCV (corresponding to the signal peptide sequences) was slightly lower at 38% and reduced further in the equivalent El sequences (approx. 25%). Distribution of basic residues in the putative GBV-B core protein revealed a high lysine/arginine content (21%) in the N-terminal region up to amino acid 85, a reduced percentage beyond this point (3.6% between amino acids 86-136) and no positively charged amino acids in the signal peptide sequence. This pattern of distribution corresponded to that present in HCV core. From these data, it was concluded that the putative GBV-B core protein shares the same domain arrangement as its counterpart in HCV.

Intracellular Localization of the GBV-B Core Protein

Previous studies on the intracellular localization of HCV core showed that the protein was directed to lipid droplets and the primary sequence determinants for this localization were present in domain 2 (19). Since the region of highest homology between the core proteins of GBV-B and HCV encompassed this domain, we expressed a N-terminal region of the GBV-B polyprotein using pSFV/GB 1-398 in which the coding region extended beyond the predicted C-terminus of El to analyze the intracellular localization of GBV-B core. As immunological reagents against GBV-B proteins were not available, we placed a short epitope tag (32) at residue 85 to detect the protein. Placing this tag into the corresponding region of the HCV core protein did not affect its intracellular localization and the protein was present on lipid droplets. Indirect immunofluorescence of cells electroporated with RNA from pSFV/GB1-398 using an antibody, 9220, that recognizes the epitope tag revealed staining around lipid droplets stained with oil red O. Western blot analysis of cell extracts indicated that the size of core protein detected was approximately 17 kDa. This is consistent with a product of about 155 amino acids (comprising 143 amino acids of GBV-B and 12 amino acids of the epitope tag).

To further verify the intracellular localization of GBV-B core and the likely events involved in its maturation, two other constructs containing GBV-B sequences were examined. Firstly, a construct pSFV/GB1-141 that would correspond approximately in size to the product detected with pSFV/GB1-398. Analysis of cells expressing the tagged product produced by pSFV/GB1-141 again revealed localization of the protein around lipid droplets. The size of the protein made by pSFV/GB1-141 was only slightly smaller than that made by pSFV/GB1-398. Another construct that expressed the N-terminal 194 residues of GBV-B gave a major product that was identical in size to that produced by pSFV/GB1-398; a second minor band of about 20 kDa represented uncleaved protein. It was concluded that, in common with HCV core, the equivalent GBV-B protein is directed to lipid droplets in tissue culture cells and cleavage of the GBV-B polyprotein to produce the mature form of core is directed by cellular peptidases. Our studies on HCV core revealed that domain 2 contained the sequences essential for lipid droplet association. Based on our sequence comparisons, this domain is present also in the equivalent GBV-B protein. Hence, we propose that the essential sequences for association with lipid droplets reside within the corresponding region of GBV-B core.

Discussion

From previous analysis and data presented in this example, it was proposed that the HCV core protein consisted of three domains (19, 39). Domain 1 corresponded to the mature core protein of flaviviruses while no sequences equivalent to domain 2 were present in either pesti- or flaviviruses. Domain 3 contained the signal peptide sequence that directs the HCV E1 glycoprotein to the ER lumen. Here, the sequence comparisons were extended to include GBV-B, a virus that is the closest known related virus to HCV in terms of sequence identity (29). From comparisons of the two viral sequences, domain 2 in HCV core was found also in the corresponding GBV-B protein. Sequence identity between the predicted amino acid sequences of HCV and GBV-B in the region containing domain 2 was higher (approx. 41%) than that in the N- and C-terminal flanking regions. This degree of similarity would imply that these domains in HCV and GBV-B might perform similar functions. Extensive mutational analysis and immunolocalization studies of the HCV core protein showed that domain 2 contained the key elements for directing the protein to lipid droplets (19). The data presented in this example indicated that the core protein of GBV-B also could associate with these lipid storage structures. Thus, we conclude that a function of these domains is to direct the core protein of the two viruses to lipid storage organelles.

From the above evidence, it was concluded that the HCV and GBV-B core proteins have similar domain configurations. Based on our data (19 and this example), domain 2 in both proteins represented sequences that directed them to lipid droplets. Comparison with mammalian proteins that associate with these structures did not identify any region in such proteins with features similar to those in domain 2.

The HCV and GBV-B core proteins each contained two closely spaced proline residues within domain 2. Substitution of these prolines in HCV core abolished lipid droplet association. We have shown previously that removal of short sequence elements of domain 2 from HCV core prevented lipid droplet association (19). Based on these findings, we suggest that the HCV and GBV-B core may be members of a family of proteins that share similar sequence characteristics for targeting to lipid droplets.

To summarize this example, in mammalian tissue culture cells, the core protein of hepatitis C virus (HCV) is located at the surface of lipid droplets, which are cytoplasmic structures that store lipid. The critical amino acid sequences necessary for this localization are in a region of core protein that is absent in flavi- and pestiviruses, which are related to HCV. From sequence comparisons, this region in HCV core was present in the corresponding protein of GB virus-B (GBV-B), another virus whose genomic sequence has significant similarity (homology as defined herein) to HCV. Expression of the putative GBV-B core protein revealed that it also was directed to lipid droplets. Extending the comparisons to mammalian cellular proteins, there were no amino acid similarities with the domains for lipid droplet association in HCV core.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

### General References

Altschul et al., 1990, J Mol Biol: 403-410.

Altschul et al., 1994, Nature Genetics 6:119-129.

Barba, G. et al., 1997. Proc. Natl. Acad. Sci. 94: 1200-1205

Clayerie & States (1993) Computers and Chemistry 17:191-201

Conry-Cantilena, 1997, Tibtech 15: 71-76

Devereux et al, 1984, Nucleic Acids Research 12: 387

Engelman et al., 1986, Ann Rev, Biophys. Chem 15: 343

Heid et al., 1998, Cell Tiss Res 294: 309-321

Moradpour, D. et al., 1996, Virology 222; 51-63

Moriya, K. et al., 1998, Nature Medicine 4; 1065-1067

Moriya, K. et al., 1997, J. Gen. Virol. 78; 1527-1531

Patton, S. and Huston, G. E., 1986, Lipids 21; 170-174

Wootton & Federhen, 1993, Computers and Chemistry 17:149-163

### References for Examples

1. Murphy, D. J., and Vance, J. (1999) Trends Biol. Sci. 24, 109-115.

2. Zwytick, D., Athenstaedt, K., and Daum, G. (2000) Biochim. Biophys Acta 1469, 101-120.

3. Murphy, D. J. (2001) Prog. Lipid Res. 40, 325-438.

4. Huang, A. H. C. (1996) Plant Physiol. 110, 1055-1061.

5. van Rooijen, G. J. H., and Moloney, M. M. (1995) Plant Physiol. 109, 1353-1361.

6. Abell, B. M., Holbrook, L. A., Abenes, M., Murphy, D. J., Hills, M. J., and Moloney, M. M. (1997) Plant Cell 9, 1481-1493.

7. Jiang, H. P., and Serrero, G. (1992) Proc. Natl. Acad. Sci. U.S.A. 89, 7856-7860.

8. Brasaemle, D. L., Barber, T., Wolins, N. E., Serrero, G., Blanchette-Mackie, E. J., and Londos, C. (1997) J. Lipid Res. 38, 2249-2263.

9. Greenberg, A. S., Egan, J. J., Wek, S. A., Garty, N. B., Blanchette-Mackie, E. J., and Londos, C. (1991) J. Biol Chem. 266; 11341-11346.

10. Greenberg, A. S., Egan, J. J., Wek, S. A., Moos, M. C. Jr., Londos, C., and Kimmel, A. R. (1993) Proc. Natl. Acad. Sci. U.S.A. 90, 12035-12039.

11. Londos, C., Brasaemle, D. L., Schultz, C. J., Segrest, J. P., and Kimmel, A. R. (1999) Cell Develop. Biol. 10, 51-58.

12. Heid, H. W., Moll, R., Schwetlick, I., Rackwitz, H. R., and Keenan, T. W. (1998) Cell Tissue Res. 294, 309-321.

13. Wang, X. K., Reape, T. J., Li, X., Rayner, K., Webb, C. L., Bumand, K. G., and Lysko, P. G. (1999) FEBS Lett. 462, 145-150.

14. Rae, F. K., Stephenson, S. A., Nicol, D. L., and Clements, J. A. (2000) Int. J. Cancer 88, 726-732.

15. Blanchette-Mackie, E. J., Dwyer, N. K., Barber, T., Coxey, R. A., Takeda, T., Rondinone, C. M., Theodorakis, J. L., Greenberg, A. S., and Londos, C. (1995) J. Lipid Res. 36, 1211-1226.

16. Servetnick, D. A., Brasaemle, D. L., Gruia-Gray, J., Kimmel, A. R., Wolff, J., and Londos, C. (1995) J. Biol. Chem. 270, 16970-16973.

17. Moradpour, D., Englert, C., Wakita, T., and Wands, J. R. (1996) Virology 222, 51-63.

18. Barba, G., Harper, F., Harada, T., Kohara, M., Goulinet, S., Matsuura, Y., Eder, G., Schaff, Zs., Chapman, M. J., Miyamura, T., and Brechot, C. (1997) Proc. Natl. Acad. Sci. U.S.A. 94, 1200-1205.

19. Hope, R. G., and McLauchlan, J. (2000) J. Gen. Virol. 81, 1913-1925

20. Baumert, T. F., Ito, S., Wong, D. T., and Liang, T. J. (1998) J. Virol. 72, 3827-3836.

21. Rice, C. M. (1996) in Fields Virology (Fields, B. N., Knipe, D. M., Howley, P. M. et al., eds) 3rd ed. Vol 1, pp.931-960, Lippincott-Raven Publishers, Philadelphia, Pa.

22. Hijikita, M., Kato, N., Ootsuyuma, Y., Nakagawa, M., and Shimotohno, K. (1991) Proc. Natl. Acad. Sci. U.S.A. 88, 5547-5551.

23. Grakoui, A., Wychowski, C., Lin, C., Feinstone, S. M., and Rice, C. M. (1993) J. Virol. 67, 1385-1395.

24. Santolini, E., Migliaccio, G., and La Monica, N. (1994) J. Virol. 68, 3631-3641.

25. Lo, S. -Y., Masiarz, F., Hwang, S. B., Lai, M. M. C., and Ou, J. -H. (1995) Virology 213, 455-461.

26. Hussy, P., Langen, H., Mous, J., and Jacobsen, H. (1996) Virology 224, 93-104.

27. Moriya, K., Yotsuyanagi, H., Shintani, Y., Fujie, H., Ishibashi, K., Matsuura, Y., Miyamura, T., and Koike, K. (1997) J. Gen. Virol. 78, 1527-1531.

28. Sabile, A., Perlemuter, G., Bono, F., Kohara, K., DeMaugre, F., Kohara, M., Matsuura, Y., Miyamura, T., Brechot, C., and Barba, G. (1999) Hepatology 30, 1064-1076.

29. Muerhoff, A. S., Leary, T. P., Simons, J. N., Pilot-Matias, T. J., Dawson, G. J., Erker, J. C., Chalmers, M. L., Schlauder, G. G., Desai, S. M., and Mushahwar, I. K. (1995) J. Virol. 69, 5621-5630.

30. Beames, B., Chavez, D., Guerra, B., Notvall, L., Brasky, K. M. and Lanford, R. E. (2000) J. Virol. 74; 11764-11772.

31. Bukh, J., Apgar, C. L., and Yanagi, M. (1999) Virology 262, 470-478.

32. McLauchlan, J., Liefkens, K., and Stow, N. D. (1994) J. Gen. Virol. 75, 2047-2052.

33. Liljestrom, P., and Garoff, H. (1991) Bio-Technology 9, 1356-1361.

34. Keddie, J. S., Hubner, G., Slocombe, S. P., Jarvis, R. P., Cummins, I., Edwards, E. W., Shaw, C. H., and Murphy, D. J. (1992) Plant Mol. Biol. 19, 443-453.

35. Sarmiento, C., Ross, J. H. E., Herman, E., and Murphy, D. J. (1997) Plant Journal 11, 783-796.

36. Patel, J., Patel, A. H., and McLauchlan, J. (1999) J. Gen. Virol. 80, 1681-1690.

37. Thompson, J. D., Higgins, D. G., and Gibson, T. J. (1994) Nucl. Acids Res. 22, 4673-4680.

38. Kyte, J., and Doolittle, R. F. (1982) J. Mol. Biol. 157, 105-132.

39. McLauchlan, J. (2000) J. Viral Hepatitis 7, 2-14.

40. Mandl, C. W., Heinz, F. X., and Kunz, C. (1988) Virology 166, 197-205.

41. Amberg, S. M., Nestorowicz, A., McCourt, D. W., and Rice C. M. (1994) J. Virol 68, 3794-3802.

42. Amberg, S. M. and Rice, C. M. (1999) J. Virol. 73, 8083-8094.

43. Nielsen, H., Engelbrecht, J., Brunak, S., and von Heijne, G. (1997) Protein Eng. 10, 1-6.

44. Ting, J. T. L., Balsamo, R. A., Ratnayake, C., and Huang, A. H. C. (1997) J Biol. Chem. 272, 3699-3706.

45. Chen, J. C. F., Tsai, C. C. Y., and Tzen, J. T. C. (1999) Plant Cell Physiol. 40, 1079-1086.

46. Naested, H., Frandsen, G. I., Jauh, G -Y., Hemandez-Pinzon, I., Nielsen, H. B., Murphy, D. J., Rogers, J. C., and Mundy, J. (2000) Plant Mol. Biol. 44, 463-476.

47. Lacey, D. J., Wellner, N., Beaudoin, F., Napier, J. A., and Shewry, P. R. (1998) Biochem. J. 334, 469-477.

48. Fujimoto, T., Kogo, H., Ishiguro, K., Tauchi, K., and Nomura, R. (2001) J. Cell Biol. 152, 1079-1085.

49. Ostermeyer, A. G., Paci, J. M., Zeng, Y., Lublin, D. M., Munro, S., and Brown, D. A. (2001) J. Cell Biol. 152, 1071-1078.

50. Pol, A., Luetterforst, R., Lindsay, M., Heino, S., Ikonen, E., and Parton, R. G. (2001) J. Cell Biol. 152, 1057-1070.

51. Li, M., Smith, L. J., Clark, D. C., Wilson, R., and Murphy, D. J. (1992) J. Biol. Chem. 267, 8245-8253.

What is claimed is:

1. A method for identifying a substance capable of affecting a viral infection, comprising:
providing a lipid globule targeting sequence, as a first component;
providing a lipid globule, as a second component;
contacting the two components with a substance to be tested under conditions that would permit the two components to interact in the absence of the substance; and
determining whether the substance disrupts the interaction between the first and second components.

2. The method of claim 1, wherein the targeting sequence comprises a hepatitis C virus (HCV) core protein or a fragment thereof, or a GB virus-B core protein or a fragment thereof.

3. The method of claim 2, wherein the targeting sequence further comprises variants or homologues of the hepatitis C virus (HCV) core protein or the GB virus-B core protein.

4. The method according to claim 1, wherein the substance to be tested is administered to a cell, the lipid globule targeting sequence is expressed in said cell and the lipid globule is a natural constituent of said cell.

5. The method according to claim 4, wherein the lipid globule targeting sequence is naturally or recombinantly expressed in said cell.

6. The method according to claim 1, further comprising:
administering a virus to a cell in the absence of said substance which has been determined to disrupt the interaction between the first and second components;
administering the virus to the cell in the presence of said substance; and
determining if said substance reduces or abolishes the susceptibility of the cell to viral infection or the effects of viral infection.

7. The method according to claim 1, wherein the lipid globule targeting sequence comprises amino acids of the HCV core protein selected from the group consisting of 125 to 144, 161 to 166 and the combination thereof.

8. The method according to claim 1, wherein the viral infection is a hepatitis infection or other viral infection of the human or animal liver.

9. The method according to claim 4, wherein said cell is a liver cell.

10. The substance identified by the method of claim 1.

11. The substance according to claim 10, wherein said substance has not previously been known to affect viral infection.

12. A method for identifying a substance for treating or preventing a viral infection, comprising:
administering said substance to a mammalian cell; and
identifying whether the administration of said substance upregulates expression of adipocyte-specific differentiation related protein (ADRP) in the mammalian cell.

13. A substance capable of disrupting an interaction between a lipid globule targeting sequence and a lipid globule for use in affecting a viral infection, wherein the targeting sequence comprises a hepatitis C virus (HCV) core protein or a fragment thereof, or a GB virus-B core protein or fragment thereof.

14. A polypeptide comprising a lipid globule targeting sequence for use in preventing or treating a viral infection, wherein the targeting sequence comprises an HCV core protein or a fragment, variant or homologue thereof, or a GB virus-B core protein or a fragment, variant or homologue thereof.

15. The polypeptide according to claim 14, wherein the targeting sequence comprises amino acids of the HCV core protein selected from the group consisting of 125 to 144, 161 to 166 and the combination thereof.

16. A pharmaceutical composition comprising the polypeptide according to claim 15 and a pharmaceutically acceptable carrier or diluent.

17. A method for treating or preventing a viral infection comprising administering an effective amount of the pharmaceutical composition of claim 16.

18. A method for treating or preventing a viral infection comprising administering an effective amount of a pharmaceutical composition comprising the substance identified by claim 1 and a pharmaceutically acceptable carrier or diluent.

19. A method for treating or preventing a viral infection comprising administering an effective amount of a pharmaceutical composition comprising the substance identified by claim 12 and a pharmaceutically acceptable carrier or diluent.

20. A polynucleotide encoding a polypeptide according to claim 14 for use in treating or preventing a viral infection.

21. A method for determining whether a test substance is capable of treating or preventing a viral infection, comprising:
providing a lipid globule targeting sequence, as a first component, said targeting sequence comprising a hepatitis C virus (HCV) core protein or a fragment thereof, or a GB virus-B core protein or fragment thereof;
providing a lipid globule, as a second component;
incubating the first and second components with the test substance under conditions that would permit the first and second components to interact with one another in the absence of the test substance; and
determining whether the test substance disrupts the interaction between the first and second components.
